(19) United States
(12) Patent Application Publication
(10) Pub. No.: US 2018/0028836 A1
(43) Pub. Date: Feb. 1, 2018

(54) RADIOACTIVE MEDICAL DEVICE

(71) Applicant: BOSTON SCIENTIFIC SCIMED, INC., Maple Grove, MN (US)

(72) Inventors: ARNOLD M. HERSKOVIC, Chicago, IL (US); CLAUDE O. CLERC, Marlborough, MA (US); JOHN THOMAS FAVREAU, Spencer, MA (US)

(21) Appl. No.: 15/660,521

(22) Filed: Jul. 26, 2017

Related U.S. Application Data
(60) Provisional application No. 62/367,805, filed on Jul. 28, 2016.

(51) Int. Cl. A61N 5/10; A61K 51/12; A61K 51/04

(52) U.S. Cl. CPC A61N 5/1001; A61K 51/0495; A61K 51/1282; A61N 5/1002

(57) ABSTRACT

This disclosure provides design, material, manufacturing method, and use alternatives for medical devices. An example medical device comprises a radioactive element positionable within a body lumen, the body lumen having an inner surface. The medical device also includes a frame attached to the radioactive element. The frame is also configured to position the radioactive element radially inward and away from the inner surface of the body lumen.

### CROSS REFERENCE TO RELATED APPLICATIONS

This application claims the benefit of U.S. Provisional Patent Application Ser. No. 62/367,805 filed on Jul. 28, 2016, the disclosure of which is incorporated herein by reference.

### TECHNICAL FIELD

The present disclosure pertains to medical devices, and methods for manufacturing medical devices. More particularly, the present disclosure pertains to radioactive elements connected with other structures, and methods for manufacturing and using such devices.

### BACKGROUND

Some cancers and neoplasms are easier to treat with radiation than others. Hard-to-reach neoplasms, such as those in the esophagus, intestines and other lumens, are often treated via Brachytherapy so as to minimize radiation to adjacent, healthy tissue.

Brachytherapy delivers radiation to small tissue volumes while limiting exposure of healthy tissue. In this regard, the delivered radiation conforms more to the target than any other form of radiation, (including proton therapy) as less normal transient tissue is treated. It features placement of radiation sources, such as small radioactive particles or needles, near or within the target tissue, thus having the advantage over External Beam Radiation Therapy (EBRT) of being more focalized and less damaging to surrounding healthy tissue.

Brachytherapy is a common treatment for esophageal, prostate, and other cancers. Brachytherapy has been used to treat prostate cancer which has been practiced for more than half a century. In this situation, very low activity material emitting a low energy is placed next to or within a tumor. Traditionally, these low emitting devices have mostly been left in place permanently except in extraordinary circumstances. It would be desirable to utilize radioactive material in conjunction with interventional medical devices when clinically appropriate, and/or it may be desirable to tailor the delivery of radioactive energy or radioactive sources according to clinical needs. For example, it may be advantageous to couple a radiation source with a frame when clinically necessary and/or it may be advantageous to adjust the position and the activity of the radioactive source on a frame in response to changes in tumor shape and size, carrier position, and other relevant therapeutic factors.

### BRIEF SUMMARY

This disclosure provides design, material, manufacturing method, and use alternatives for medical devices. An example medical device comprises a radioactive element positionable within a body lumen, the body lumen having an inner surface. The medical device also includes a frame attached to the radioactive element. The frame is also configured to position the radioactive element radially inward and away from the inner surface of the body lumen.

Alternatively or additionally to any of the embodiments above, the frame is configured to position the radioactive element in a central region of the body lumen.

Alternatively or additionally to any of the embodiments above, the device further includes a plurality of radioactive elements, wherein the plurality of radioactive elements are attached together to form an elongated radioactive strand.

Alternatively or additionally to any of the embodiments above, the radioactive element is removably attached to the frame.

Alternatively or additionally to any of the embodiments above, the frame includes one or more support arms extending radially away from the radioactive element.

Alternatively or additionally to any of the embodiments above, the one or more support arms each include a fixation member positioned on an end thereof.

Alternatively or additionally to any of the embodiments above, each of the one or more support arms further comprises a spring member attached thereto.

Alternatively or additionally to any of the embodiments above, each of the one or more support arms are configured to be releasably engaged to the body lumen.

Alternatively or additionally to any of the embodiments above, the frame includes a retrieval member, and wherein pulling the retrieval member collapses each of the one or more support arms toward the radioactive element.

Alternatively or additionally to any of the embodiments above, the device further includes an anchoring member positioned around the radioactive element, and wherein each of the one or more support arms include a first end secured to the anchoring member.

Alternatively or additionally to any of the embodiments above, the second ends of the one or more support arms are axially aligned.

Alternatively or additionally to any of the embodiments above, each of the one or more support arms are configured to permit the radioactive element to shift from a first position to a second position.

Alternatively or additionally to any of the embodiments above, shifting the support arms from a first position to a second position shifts the radioactive element in a radial direction, an axial direction, or both radial and axial directions.

Another example medical device comprises a support structure including a base member and one or more support members extending therefrom. The base member is configured to receive one or more radioactive elements. The base member is also configured to position the one or more radioactive elements in a central region of a body lumen.

Alternatively or additionally to any of the embodiments above, the one or more radioactive elements are configured to be removed from the support structure.

Alternatively or additionally to any of the embodiments above, each of the one or more support members are configured to be releasably engaged to the body lumen.

Alternatively or additionally to any of the embodiments above, the device further includes an anchoring member positioned around the support structure, and wherein each of the one or more support members include a first end secured to the base member and a second end secured to the anchoring member.

Alternatively or additionally to any of the embodiments above, each of the one or more support members are configured to permit the base member to shift from a first position to a second position.

Alternatively or additionally to any of the embodiments above, shifting the one or more support members from a first position to a second position shifts the base member in both a radial direction, an axial direction, or both radial and axial directions.

Another medical device comprises a radioactive element and an expandable scaffold including a base member and one or more support members extending radially from the base member. Each of the one or more support members has a first end attached to the scaffold and a second end attached to the base member. Further, the base member is configured to receive the radioactive element and the one or more support members is/are configured to suspend the base member in a central region of a body lumen.

The above summary of some embodiments is not intended to describe each disclosed embodiment or every implementation of the present disclosure. The Figures, and Detailed Description, which follow, more particularly exemplify these embodiments.

### BRIEF DESCRIPTION OF DRAWINGS

The disclosure may be more completely understood in consideration of the following detailed description in connection with the accompanying drawings, in which:

FIG. 1 is an example radioactive element and frame.

FIG. 2 is an example radioactive element.

FIG. 3 is an example radioactive strand having radioactive seeds and spacers.

FIG. 4 is a cross section of a radioactive element and frame taken along line 4-4 of FIG. 1.

FIG. 5 is a cross section of another example radioactive element and frame.

FIG. 6 is another example radioactive element and frame.

FIG. 7 is another example radioactive element and frame.

FIG. 8 is another example radioactive element and frame.

FIG. 9 is another example radioactive element and frame.

FIG. 10 is another example radioactive element and frame positioned in a body lumen.

FIG. 11 is another example radioactive element and frame positioned in a body lumen.

FIG. 12 is a cross section of the radioactive element and frame taken along line 12-12 of FIG. 11.

FIG. 13 is an alternative cross section of an example radioactive element and frame taken along line 12-12 of FIG. 11.

FIG. 14 is another example radioactive element and frame positioned in a body lumen.

FIG. 15 is another example radioactive element and frame positioned in a body lumen.

FIG. 16 is a cross section of the radioactive element and frame taken along line 16-16 of FIG. 15.

FIG. 17 is an alternative cross section of a radioactive element and frame taken along line 16-16 of FIG. 15.

FIG. 18 is another example radioactive element and frame positioned in a body lumen.

FIG. 19 is a cross section of the radioactive element and frame taken along line 19-19 of FIG. 18.

FIG. 20 is a cross-section of an example medical device delivery system.

FIG. 21 is a cross section of the radioactive element and frame taken along line X-X of FIG. 18.

FIG. 22 is a cross section of another example radioactive element and frame.

FIG. 23 is a cross section of another example radioactive element and frame.

FIG. 24 is a cross section of another example radioactive element and frame.

FIG. 25 is another example radioactive element and frame positioned in a body lumen.

FIG. 26 is another example radioactive element and frame positioned in a body lumen.

FIG. 27 is another example radioactive element and frame positioned in a body lumen.

FIG. 28 is another example radioactive element and frame positioned in a body lumen.

FIG. 29 is a cross-section of another example radioactive element and frame.

FIG. 30 is a cross-section of another example radioactive element and frame.

FIG. 31 is a cross-section of another example radioactive element and frame.

FIG. 32 is a cross-section of another example radioactive element and frame.

FIG. 33 is a perspective view of an example stent.

FIG. 34 is a perspective view of the stent member shown in FIG. 33.

FIG. 35 is a perspective view of the stent member shown in FIG. 33.

FIG. 36 is a perspective view of the stent member shown in FIG. 33.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the terms “distal” or “distally” are referents to a direction away from an operator of the devices of the present disclosure, while the terms “proximal” or “proximally” are referents to a direction toward the operator of the devices of the present disclosure.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Treatment of abnormal tissue growth (e.g. cancer) may be accomplished through a variety of methodologies. For example, treatment of cancer may include the placement and deployment of a radioactive element adjacent to the diseased tissue. However, in some instances treatment outcomes may be improved by tailoring one or more conventional therapies. For example, positioning radioactive elements in a particular location relative to diseased tissue may improve cancer treatment outcomes as compared to either stent or radiation therapy alone. Therefore, it may be desirable to utilize materials and/or design a medical device that permits radioactive elements to be positioned in specific locations relative to diseased tissue. Some of the examples and methods disclosed herein may include a medical device that can position radioactive elements within a central region of a body lumen.

Medical devices disclosed herein may treat esophageal cancers. Additionally, the medical device may treat other forms of disease, including gastrointestinal, airway, urethra, ureter, cardiac, brain, breast, bladder, kyphoplasty and peripheral vascular disease, for example. Further, the medical devices disclosed herein may also be used in excisional cavities in solid and/or hollow organs.

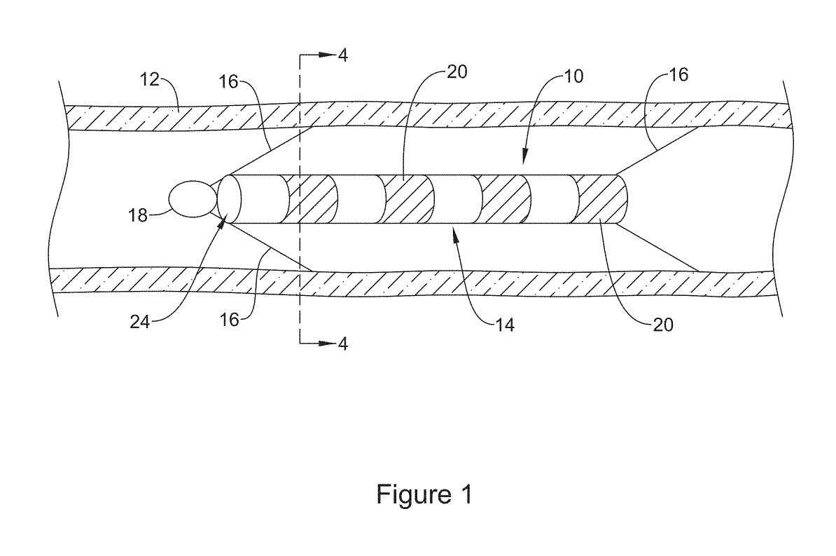

FIG. 1 shows an example radioactive device 10 positioned within an example body lumen 12. Radioactive device 10 may include one or more radioactive elements 20 secured to an example frame 14. In some examples, radioactive device 10 may further include one or more support members 16 (e.g., arms) extending away from frame 14. Additionally, radioactive device 10 may include a retrieval member 18 attached to an end region of radioactive device 10.

As stated above, in at least some examples one or more radioactive elements 20 may be secured to example frame 14. Further, it is contemplated that frame 14 may include a variety of designs, all of which may secure radioactive elements 20 along at least a portion of frame 14. It is noted that for purposes of this disclosure, the term “frame” may be defined as a base, base member, housing, framework, support structure, scaffold, tubular member, reservoir, receptacle, etc. It is further noted that for purposes of this disclosure, “securing” radioactive elements to frame 14 may include attaching, coupling, fixing, receiving, holding, maintaining, containing, and/or disposing radioactive elements 20 along a portion of frame 14 in a removable or permanent manner.

In some instances, frame 14 may include a wire and/or mesh framework (e.g., scaffold) designed to secure radioactive elements 20 thereupon. In some examples, frame 14 may substantially surround radioactive elements 20, providing a lumen or cavity to receive radioactive elements 20 therein. In other examples, frame 14 may only extend around a portion of radioactive elements 20, providing a securement region for receiving radioactive elements therealong. It can be appreciated that wire frame structure 14 may include a variety of different geometries, patterns, configurations and/or designs configured to secure radioactive elements 20 thereupon.

In some examples frame 14 may be designed such that it forms a tubular structure. For example, frame 14 may include a tubular member having a lumen 24 extending therein. It can be appreciated that radioactive elements 20 may be positioned inside at least a portion of a tubular frame 14. In some instances, a proximal end of lumen 24 may be open to receive radioactive elements 20 therein while a distal end of lumen 24 may be closed or blocked to prevent radioactive elements 20 from exiting the distal end of lumen 24.

Frame 14 may be constructed from a variety of materials. For example, frame 14 may be constructed from a metal (e.g., Nitinol, stainless steel, etc.). In other instances, frame 14 may be constructed from a polymeric material (e.g., PET, polyamide, PEEK, etc.). In yet other instances, frame 14 may be constructed from a combination of metallic and polymeric materials. Additionally, frame 14 may include a bioabsorbable and/or biodegradable material, if desired.

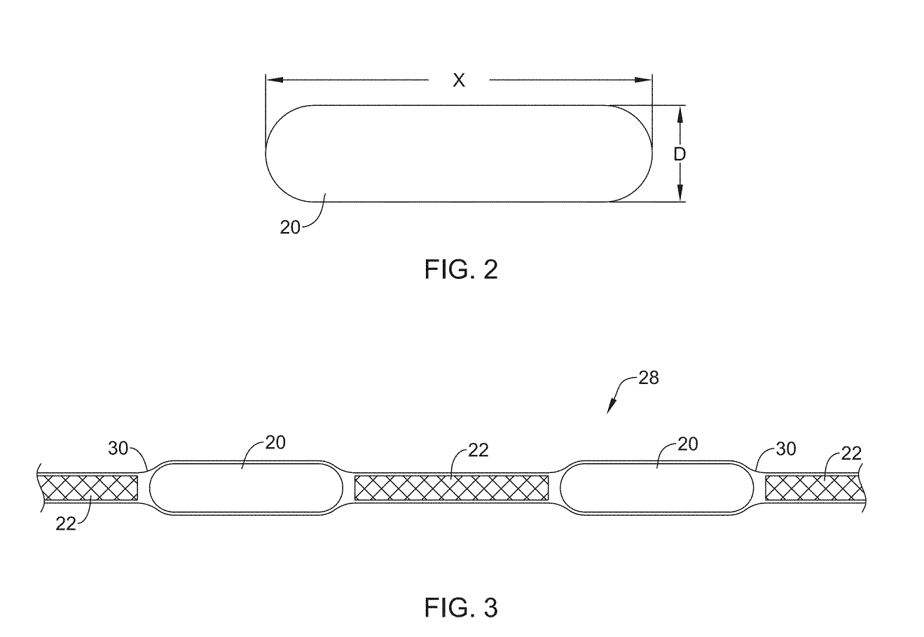

FIG. 2 shows an example radioactive element 20 which may be secured to example frame 14. In some instances, radioactive element 20 may be referred to as a “seed.” The terms “radioactive element” and “seed” may be used interchangeably throughout the remainder of this discussion. In general, seed 20 may be positioned adjacent a target site, whereby seed 20 may release radioactive energy and/or material, thereby radioactively treating the target location.

Seed 20 may be generally shaped as shown in FIG. 2. In other words, seed 20 may be an elongated cylinder having rounded ends. However, other shapes are contemplated. For example, seed 20 may be spherical, ovular, rectangular, triangular, or the like.

FIG. 2 shows the length of seed 20 depicted as dimension “X” and the diameter of seed 20 as dimension “D.” Depending on the particular therapeutic application, different types of seeds may have different dimensions. For example, in some instances, seed 20 may have a length “X” of between 1 and 20 mm. In other examples, seed 20 may have a length “X” between 2 and 10 mm, or between 3 and 8 mm. In some examples, seed 20 may have a length of about 4.5 mm.

Additionally, in some instances, seed 20 may have a diameter “D” of between 0.1 and 1.5 mm. In other examples, seed 20 may have a diameter “D” between 0.2 and 1 mm, or between 0.3 and 0.8 mm. In some examples, seed 20 may have a diameter of about 0.5 mm.

Seed 20 may include a variety of radioactive materials and or combinations of various materials. For example, seed 20 may include Iodine-125 (e.g. GE Oncura THINSeed™, IsoAid Advantage™ by IsoAid, Best™ Iodine-125), Palladium-103 (e.g. CivaString™ by CivaTech Technology, Theraseed™ by Theragenics, Best™ Palladium-103), Cesium-131, Gold-198, Iridium-192 and/or Ytterbium-169 or any other variations and/or derivatives thereof. Further, seed 20 may include other types of radioactive material. Additionally, seed 20 may include beta-emitting radionuclides.

In some instances, one or more seeds 20 may combined with one or more additional seeds 20 and/or one or more spacing elements to form an elongated treatment member 28. For example, FIG. 3 shows elongated treatment member 28 including seeds 20 and spacing elements 22 positioned between adjacent seeds 20. In some instances (including the following discussion herein), treatment member 28 may be referred to as a “strand.” In some instances, treatment member 28 (i.e., strand) may include a plurality of seeds 20 arranged adjacent to one another and/or spaced away from one another, without spacing elements 22 therebetween.

The example shown in FIG. 3 depicts a covering 30 surrounding the seeds 20 and spacers 22. In some instances, covering 30 may include a material capable of being placed over the combination of seeds 20 and/or spacers 22 to form a continuous strand 28. In some examples, covering 30 may be a tubular sleeve having an inner diameter in an equilibrium state less than the diameter of seeds 20 and/or spacers 22. Accordingly, placement of seeds 20 and/or spacers 22 in the tubular sleeve expands regions of covering 30 surrounding seeds 20 and/or spacers 22. In some examples, covering 30 may include one or more of a variety of shrink tubing (e.g. a polymeric tubing capable of reducing in size upon the application or heat, for example). In other examples, the covering may include a bioabsorbable and/or biodegradable material. Additionally, in some instances seeds 20 and/or spacers 22 may be connected to one another via a bioabsorbable connector. In other words, a combination of seeds 20 and/or spacers 22 may be “linked” to one another by a bioabsorbable and/or biodegradable material. In some instances, the radioactive strand 28 may include a radioactive wire.

Seeds 20 and spacers 22 may be spaced and/or distributed in various patterns and/or distributions along strand 28. The length of the spacers 22 (which may correspond to the space between any two seeds 20) may vary depending on the particular strand 28 configuration. Similarly, the length of a given seed 20 in combination with a variety of lengths of given spacers 22 may vary depending on a particular strand 28 configuration. For example, it is contemplated that a given strand 28 may combine seeds 20 and/or spacers 22 in a variety of different combinations, patterns, distributions, separations, arrangements, or the like depending on the particular strand design required for a particular therapeutic application or user preference, for example.

For purposes of this disclosure, it is understood that radioactive elements 20 may include any of the variations of the radioactive seeds 20 and/or strands 28 discussed above. For example, frame 14 may be secured to any of the example seeds 20 and/or strands 28 discussed herein. In some examples, radioactive seeds 20 may be connected together to form a flexible or rigid elongate member (e.g., a rod).

In some instances, radioactive elements 20 may be removably secured to frame 14 such that radioactive elements 20 may be replaced with new radioactive elements in situ. For example, frame 14 may be initial implanted in body lumen 12 with radioactive elements 20. Over a period of time in which radioactive elements 20 decay, it may be desirable to replace radioactive elements 20 with new radioactive elements to continue and/or alter the treatment to the target site of body lumen 12. Accordingly, radioactive elements 20 may be removed from frame 14 while frame 14 remains implanted in body lumen 12 at the treatment site and new radioactive elements 20 delivered to the treatment site and secured to frame 14.

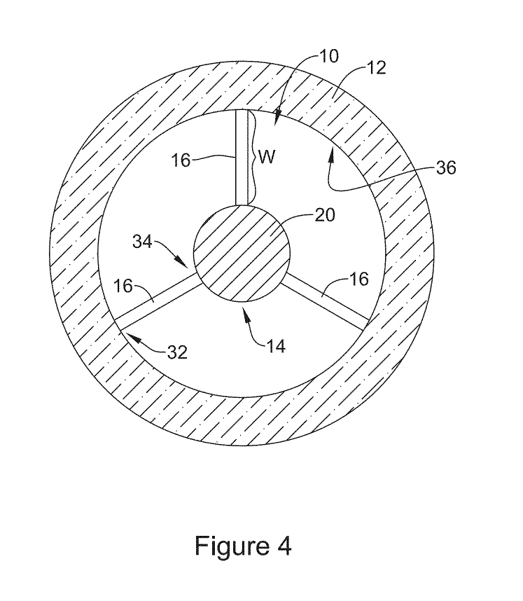

As discussed above, radioactive device 10 may include one or more support members or arms 16, or a plurality of support arms 16 designed to position device 10 at a particular location within a body lumen. For example, in some instances it may be desirable to position radioactive elements 20 in a central region of an example body lumen, such as near the central longitudinal axis of the body lumen. FIG. 4 shows a cross-section of medical device 10 along line 4-4 of FIG. 1. As illustrated in FIG. 4, medical device 10 may include three support arms 16 extending radially away from frame 14. Support arms 16 may include a first end 34 secured to frame 14 and a second end 32 engagable to an inner surface 36 of body lumen 12. Thus, support arms 16 may extend radially outward from frame 14 toward inner surface 36 of body lumen 12. In some instances, the support arms 16 may extend radially outward at an oblique angle to the central longitudinal axis of frame 14. For example, support arms 16 may extend radially outward from frame 14 in a distal direction at an oblique angle, such that support arms 16 prevent distal migration of device 10 in a body lumen. In other instances, support arms 16 may extend radially outward from frame 14 in a proximal direction at an oblique angle. In other instances, support arms 16 may extend radially outward at a substantially perpendicular angle (i.e., 90°±5°, 90°±3°, or 90°±1°) to the central longitudinal axis of frame 14.

It can be appreciated that three support arms 16 shown in FIG. 4 (spaced substantially equidistant around frame 14) may position radioactive element 20 (shown in FIG. 4 secured to frame 14) substantially in a central region of the body lumen 12. However, it can be appreciated that radioactive device 10 may include more or less than three support members 16. For example, radioactive device may include 1, 2, 3, 4, 5, 6, 7, 8, 9, 10 or more support members 16. Furthermore, it can be appreciated that support members 16 may be spaced around example frame 14 in a variety of configurations and/or arrangements.

FIGS. 1 and 4 depict support arms 16 having a length (depicted as “W” in FIG. 4) extending between frame 14 and the inner surface 36 of body lumen 12. Further, in FIGS. 1 and 4, each of the support arms 16 are shown to extend an equidistance between frame 14 and the inner surface 36 of body lumen 12, thereby positioning radioactive elements 20 in the central region of body lumen 12, such as centrally positioning the radioactive elements 20 equidistant to the perimeter of the inner surface 36 of the body lumen 12 (e.g., centering the radioactive elements 20 along the central longitudinal axis of the body lumen 12). In some examples, the distance “W” may be between 0.1 mm and 30 mm, or between about 1 mm and 20 mm, or between about 3 mm and 20 mm.

In at least some examples, it is further contemplated that one or more of support arms 16 may have differing lengths. Therefore, it can be appreciated that radioactive elements 20 may be positioned in a variety of locations within lumen 12 by altering the lengths, angular orientation from the longitudinal axis of frame 14 and/or body lumen 12 and/or arrangements of various support arms 16. For example, by designing one or more of the support arms 16 to have a length different than other support arms 16, the radioactive elements 20 may be positioned “off center” in body lumen 12. In other words, it is contemplated that altering the dimensions of support arms 16 may shift the radioactive element away from the central longitudinal axis of the body lumen 12 and closer to the inner surface 36 of body lumen 12 on one side of the central longitudinal axis than on an opposite side of the central longitudinal axis of the body lumen.

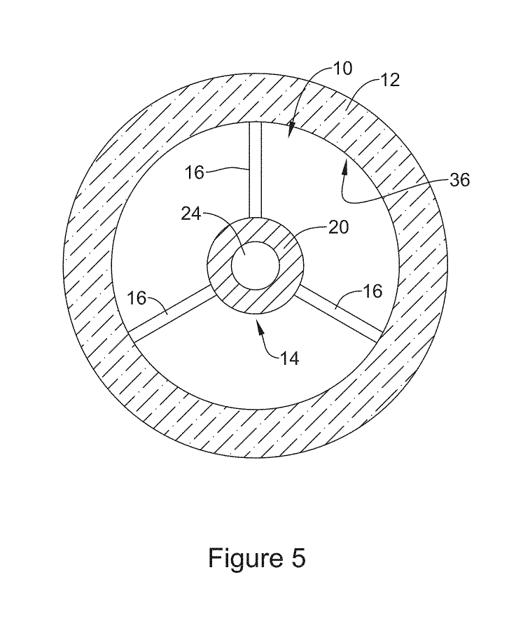

In some instances, it may be desirable for the radioactive device 10 disclosed in the above examples to be inserted into body lumen 12 over a guidewire. FIG. 5 shows that in some examples frame 14 and/or radioactive elements 20 may include a lumen 24 through which a guidewire may be inserted such that the radioactive device 10 may be advanced to a treatment location within body lumen 12 along a guidewire. It can be appreciated that lumen 24 may extend through each radioactive element 20 and/or the frame 14 along the entire axial length of medical device 10, in some instances. In other instances, the frame 14 may include a guidewire lumen offset from radioactive elements 20 through which a guidewire may be disposed.

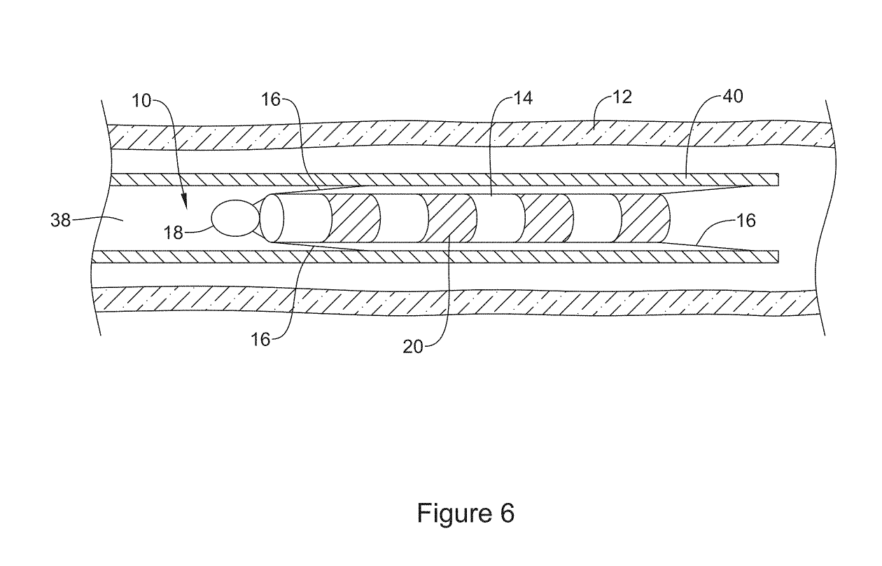

FIG. 6 illustrates medical device 10 (including frame 14, support arms 16 and radioactive elements 20) positioned within the lumen 38 of an example delivery catheter 40. As shown in FIG. 6, it is contemplated that support arms 16 may be able to flex, bend, rotate and/or collapse in a radial direction, an axial direction or both a radial and axial direction relative to frame 14 and/or radioactive elements 20. For example, FIG. 6 shows support arms 16 collapsed to fit within lumen 38 of catheter member 40.

It is further contemplated that in any of the examples disclosed herein, support arms 16 may include one or more structural elements that allow it to flex, bend, rotate and/or collapse in a radial direction, an axial direction or both a radial and axial direction relative to frame 14 and/or radioactive elements 20 for delivery and/or retrieval. For example, support arms 16 may include one or more spring elements (not shown) which permit support arms 16 to bend in a variety of directions, moving support arms 16 closer to the central longitudinal axis of frame 14. Further, the spring elements may allow the support arms 16 to shorten or lengthen relative to frame 14 and/or radioactive elements 20.

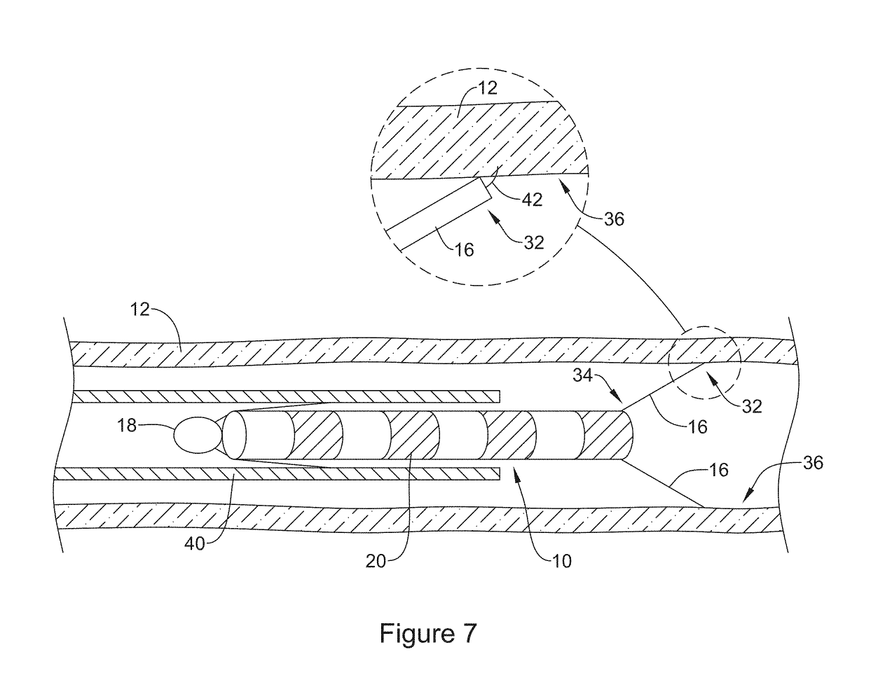

In some examples, support arms 16 may be biased to shift from the collapsed position (shown in the delivery catheter of FIG. 6) to a deployed position. For example, FIG. 7 shows a portion of device 10 (including frame 14, support arms 16 and radioactive elements 20) positioned within the lumen 38 of an example delivery catheter 40. However, FIG. 7 further shows a portion of device 10 which has been advanced out of the end of delivery catheter 40. As shown in FIG. 7, support arms 16 which have been advanced out of catheter 40 have flexed radially away from frame 14 (and radioactive elements 20) to a position in which they contact the inner surface 36 of body lumen 12. It can be appreciated that that support arms 16 may be designed with this “outward bias” such that they may expand radially away from frame 14 as medical device 10 is deployed out of an example delivery system (e.g., delivery catheter 40). In other words, support arms 16 may be configured to expand, extend and/or deflect radially outward from frame 14 when unconstrained.

Additionally, FIG. 7 includes a detail view illustrating an end region 32 of support arm 16 contacting the inner surface 36 of body lumen 12. As shown in FIG. 7, the end region 32 of support arm 16 may include an attachment member 42. Attachment member 42 may include a hook, extension, arm, fastener, projection, prong, spur or similar structure designed to grip the inner surface 36 of body lumen 12. For example, the detailed view in FIG. 7 shows attachment member 42 piercing the inner surface 36 of body lumen 12, and thus penetrating into the wall of body lumen 12 to anchor medical device 10 to body lumen 12. It can be appreciated that when attachment member 42 penetrates (e.g., pierces) the inner surface 36 of body lumen 12, medical device 10 may have an increased ability to maintain its position relative to a target site and/or body lumen 12 to prevent migration of device 10 in body lumen 12.

Further, in some examples, attachment member 42 may be designed to be releasably attached to the inner surface 36 of body lumen 12. In other words, in some examples medical device 10 may be designed such that attachment arms 16 may be deployed and thereby attachment members 42 may be inserted into the wall of body lumen 12. After deployment, attachment arms 16 may be retracted (e.g., collapsed, withdrawn, etc.), thereby removing attachment members 42 from the wall of body lumen 12 and allowing medical device to be captured within a delivery and/or retrieval device for repositioning within or removal from a patient's body lumen. In other instances, attachment arms 16 may be detached from support arms 16 to permit removal of medical device 10 from body lumen 12.

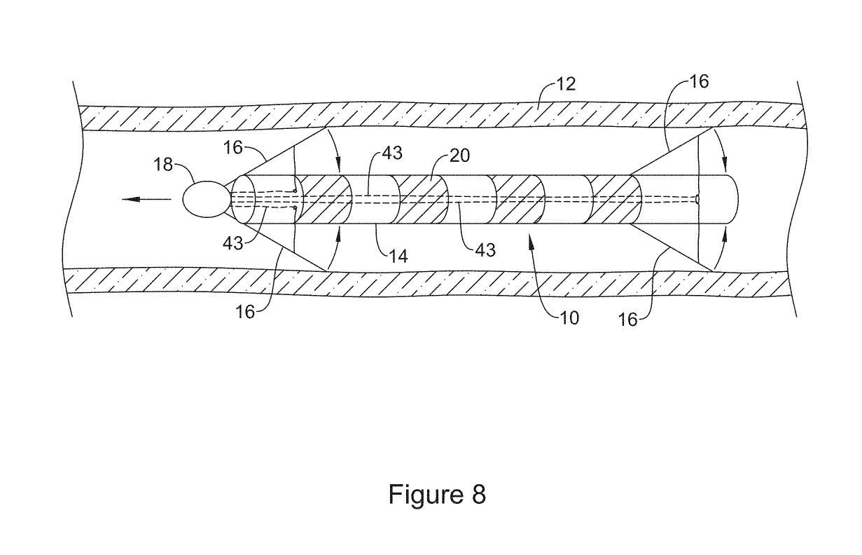

FIG. 8 illustrates an example radioactive medical device 10 (including frame 14, support arms 16 and radioactive elements 20) designed to be removed from and/or repositioned within example body lumen 12. As shown in FIG. 8, example medical device 10 includes one or more tethers 43 extending from retrieval member 18 to each of the attachment arms 16. In some examples, tethers 43 may include wires, strings, etc. that extend along or are integrated with frame 14 and/or radioactive elements 20.

As shown in FIG. 8, tethers 43 may include a first end that may be attached and/or coupled to retrieval member 18 and a second end that is attached and/or coupled to attachment members 16. It can be appreciated that pulling retrieval member 18 (as depicted by the arrow parallel to body lumen 12 in FIG. 8) may pull tethers 43, which in turn may pull attachment arms 16 inward toward frame 14 and radioactive elements 20 (as depicted by the curved arrows in FIG. 8). It can be appreciated that while FIG. 8 shows tethers 43 positioned parallel and orthogonal to frame 14, a variety of different designs may be implemented within the design of medical device 10 to permit retrieval member 18 to collapse attachment arms 16 inwardly toward frame 14 via tethers 43.

In some instances, it may be desirable to remove and or replace radioactive elements 20 from frame 14. For example, in some instances it may be desirable for a clinician to replace radioactive elements that have decayed to levels which are no longer beneficial to the treatment of diseased tissue and/or replace radioactive elements for a modified medical treatment.

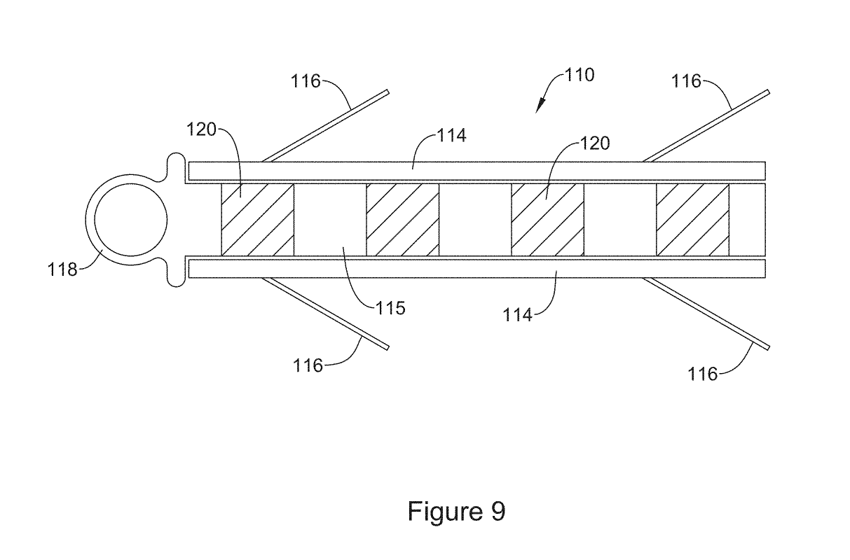

FIG. 9 illustrates an example radioactive medical device 110 including attachment arms 116 secured to frame member 114. The attachment arms 116 and frame 114 shown in FIG. 9 may be designed and operate similarly to the attachment arms 16 and frame 14 discussed above with respect to FIGS. 1-8. Additionally, medical device 110 includes radioactive elements 120 secured to removable and/or replaceable cartridge member 115. For example, cartridge member 115 may be a tubular member within which radioactive elements 120 may be secured. However, it is contemplated that cartridge member 115 may include a variety of structures designed to secure radioactive elements 120.

It can be appreciated from FIG. 9 that cartridge 115 may be removed (e.g., separated) from frame 114. In other words, cartridge 115 may be designed to mate with frame 114 such that cartridge 115 may slide and/or insert into frame 114. Additionally, medical device 110 may include a retrieval member 118 attached to cartridge 115. In some examples, retrieval member 118 may form a unitary member with cartridge 115. Retrieval member 118 may be designed to allow a clinician to grasp and manipulate cartridge 115 with a secondary medical instrument. It can be appreciated that frame member 114 may remain in a body lumen while cartridge 115 is removed and re-inserted within frame 114 upon replacing radioactive elements 20 in cartridge 115 with new radioactive elements 20, or removed and replaced with another cartridge 115 containing new radioactive elements 20.

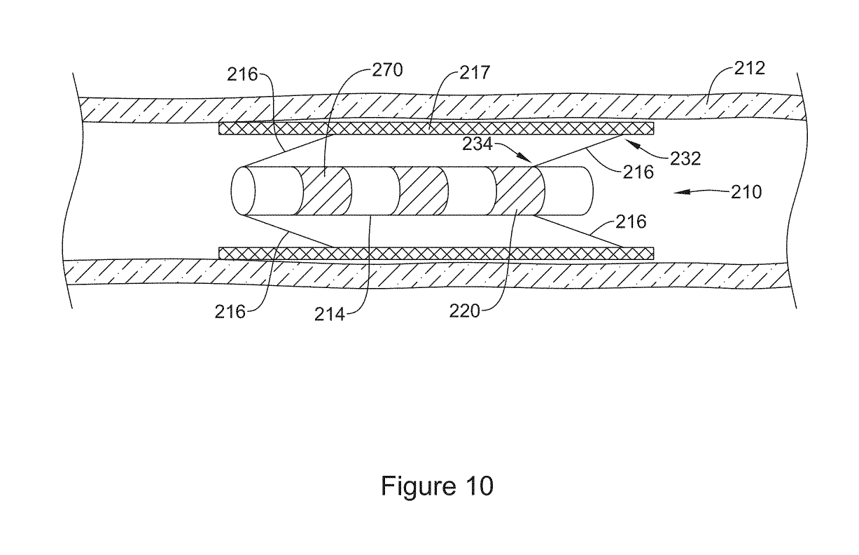

FIG. 10 illustrates another example radioactive medical device 210. Radioactive medical device 210 may include frame member 214, support members 216 and radioactive elements 220 as described above with respect to FIGS. 1-9. Additionally, FIG. 10 illustrates an anchoring member 217 that substantially surrounds at least a portion of medical device 210. In some instances, anchoring member 217 may surround all of medical device 210.

Anchoring member 217 may define a variety of designs and or structures. For example, anchoring member 217, such as a framework or scaffold, which may be an expandable framework or expandable scaffold in some instances, may include a stent, such as an expandable stent, a self-expanding stent, or another endoprosthesis or tubular member, for example. In some instances, anchoring member 217 may be manufactured from a single, cylindrical tubular member. For example, in some instances, a cylindrical tubular member may be laser cut to form an expandable stent. Anchoring member 217 may include one or more struts arranged in various designs and/or patterns. For example, anchoring member 217 may be a laser cut stent formed from a unitary tubular member. Therefore, numerous designs, patterns and/or configurations for the stent cell openings, strut thicknesses, strut designs, stent cell shapes are contemplated and may be utilized with embodiments disclosed herein. In other instances, anchoring member 217 may be a woven, braided or knitted tubular member, such as an expandable stent (e.g., self-expanding stent) formed from one or more, or a plurality of interwoven wire filaments.

As shown in FIG. 10, attachment arms 216 may extend away from radioactive elements 220 and attach to anchoring member 217. For example, each of attachment arms 216 may include a first end 234 secured to frame member 214 and/or radioactive elements 220 and a second end 232 attached to anchoring member 217.

It can be appreciated that medical device 210 (including frame member 214, support members 216, radioactive elements 220 and anchoring member 217) may be delivered to a target site in example body lumen 212 via a delivery system similar to that described with respect to FIGS. 6 and 7. However, medical device 210 may further include collapsing anchoring member 217 (in addition to frame member 214, support members 216 and radioactive elements 220) collapsed inside a delivery catheter. It can be appreciated that in some examples that as medical device 210 is deployed out of a delivery catheter, the anchoring member 217 may self-expand, thereby expanding (e.g., flexing outward) support arms 216 and positioning frame 217 and radioactive elements 220 in a central region of body lumen 212, such as near the central longitudinal axis of body lumen 212. For instance, each of the support arms 216 may extend an equidistance between frame member 214 and the anchoring member 217 such that frame member 214 (and radioactive elements 220) is centered along the central longitudinal axis of anchoring member 217, thereby positioning radioactive elements 220 in the central region of anchoring member 217 and body lumen 212, such as centrally positioning the radioactive elements 220 equidistant to the perimeter of the inner surface of the body lumen 212 (e.g., centering the radioactive elements 220 along the central longitudinal axis of anchoring member 217 and the body lumen 212).

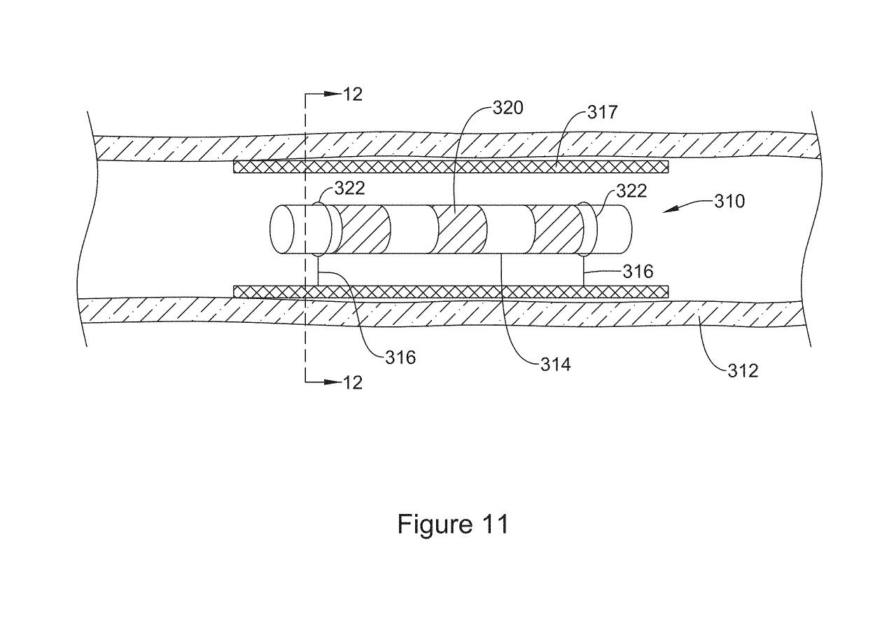

FIG. 11 illustrates another example radioactive medical device 310. As shown in FIG. 11, radioactive medical device 310 includes a frame 314, radioactive elements 320 and anchoring member 317 which may be similar in structure to the frame, radioactive elements, and the anchoring member described in the examples above. However, FIG. 11 further shows attachment arms 316 extending between anchoring member 317 and frame 314.

Attachment arms 316 may be similar in structure to the attachment arms described in the examples above. However, in some examples attachment arms 316 may further include a securement structure 322 that substantially surrounds frame 314 and radioactive elements 320. In some examples, securement structure 322 may include an eyelet, loop, hoop, ring, etc. that forms an aperture through which frame and/or radioactive elements 320 may extend. As described above (and shown in FIG. 11), attachment arms 316 may be designed such that they position, suspend, locate, etc. frame 314 (including radioactive elements 320) in a central region of anchoring member 317 and example body lumen 312, such as near the central longitudinal axis of body lumen 312. For instance, attachment arms 316 may extend radially outward from frame 314 to anchoring member 317 such that frame 314 (and radioactive elements 320) is centered along the central longitudinal axis of anchoring member 317, thereby positioning radioactive elements 320 in the central region of anchoring member 317 and body lumen 312, such as centrally positioning the radioactive elements 320 equidistant to the perimeter of the inner surface of the body lumen 312 (e.g., centering the radioactive elements 320 along the central longitudinal axis of anchoring member 317 and the body lumen 312).

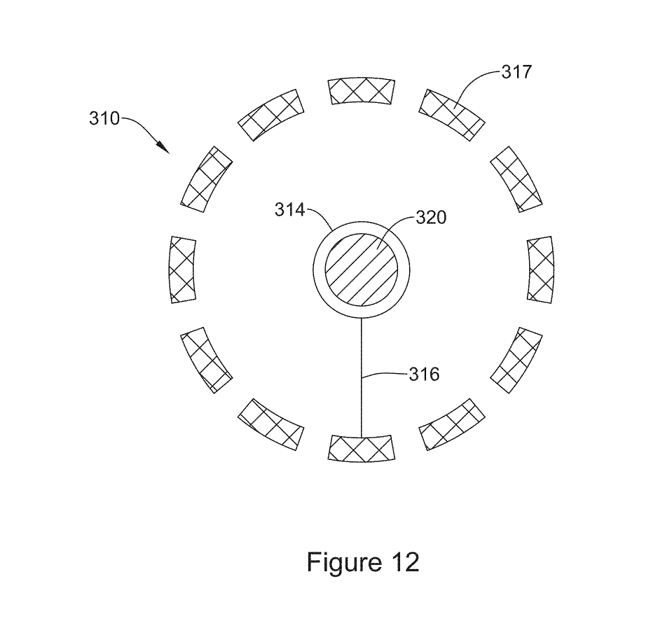

Additionally, FIGS. 11 and 12 illustrate that in some examples, radioactive medical device 310, may include a plurality of attachment arms 316, such as two attachment arms 316 that may be aligned along the longitudinal axis of medical device 310. For example, FIG. 12 is a cross-section along line 12-12 of FIG. 11 illustrating that each attachment arm 316 may include an attachment point along anchoring member 317, whereby the two attachment points may be aligned longitudinally.

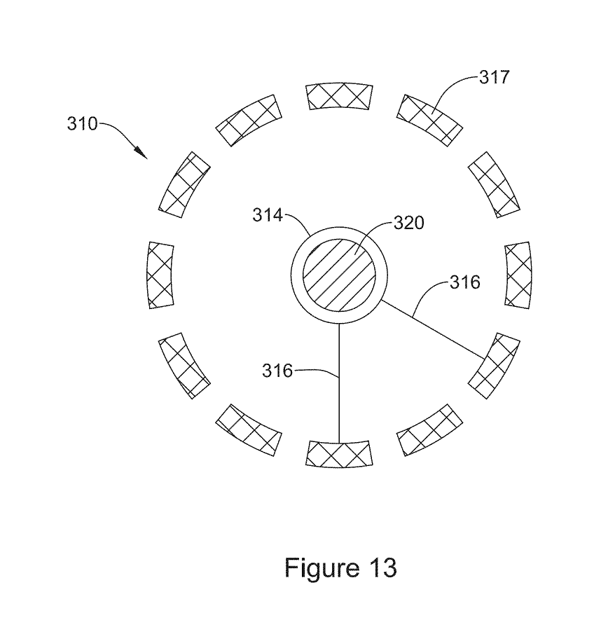

FIG. 13 is an example cross-section illustrating that in at least some examples contemplated herein medical device 310 may include more than one attachment arm 316 extending between anchoring member 317 and frame 314. It can be appreciated that multiple attachment arms 316 may be arranged in a variety of configurations. For example, in some examples, two or more attachment arms 316 may extend away from anchoring member 317 and secure to frame 314 at a single point. In other examples, multiple attachment arms 316 may be positioned at various points along the longitudinal axis of medical device 310 and be secured to frame 314 at various points along the longitudinal axis of frame 314. Furthermore, it is contemplated that in some examples, one or more attachment arms 316 may be longitudinally aligned (as shown in FIGS. 11-13. However, it is contemplated that in other examples one or more attachment arms may not be longitudinally aligned.

Additionally, while the examples disclosed above may depict radioactive medical device as including two or more attachment arms, it is contemplated that any of the examples disclosed herein may include only a single attachment arm extending between either the body lumen or anchoring member and the frame (including radioactive elements).

In some instances, it may be desirable for a radioactive medical device to shift from a position in a central region of body lumen to a position in which the medical device is closer to the inner surface of the body lumen. For example, in some instances it may be desirable for medical device 310 (shown in FIG. 11) to be able to shift in response to bodily material (e.g., food, vomit, etc.) moving through the esophagus or other body lumen within which the medical device 310 is positioned. For example, in any of the examples described herein, attachment arms coupling a frame member to either the body lumen or an anchoring member may include one or more deflectable elements which may allow the attachment arms to shift in a variety of directions.

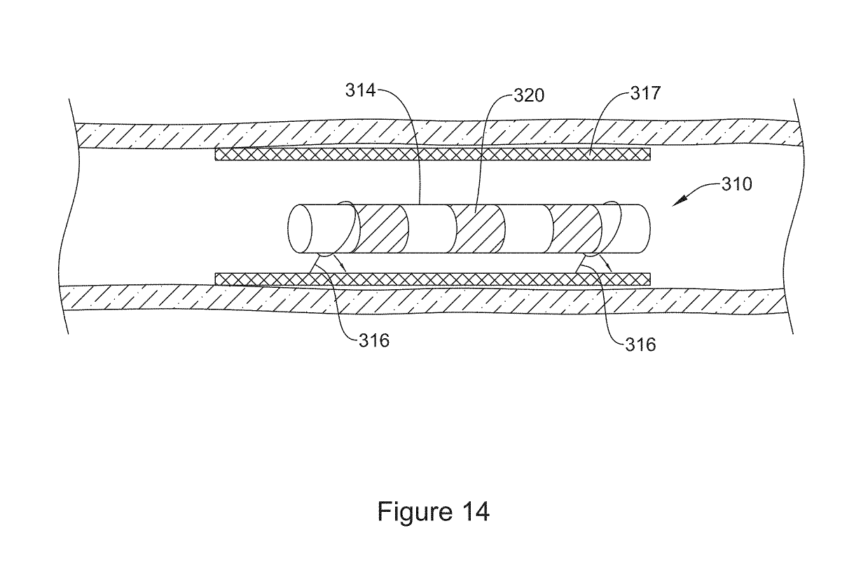

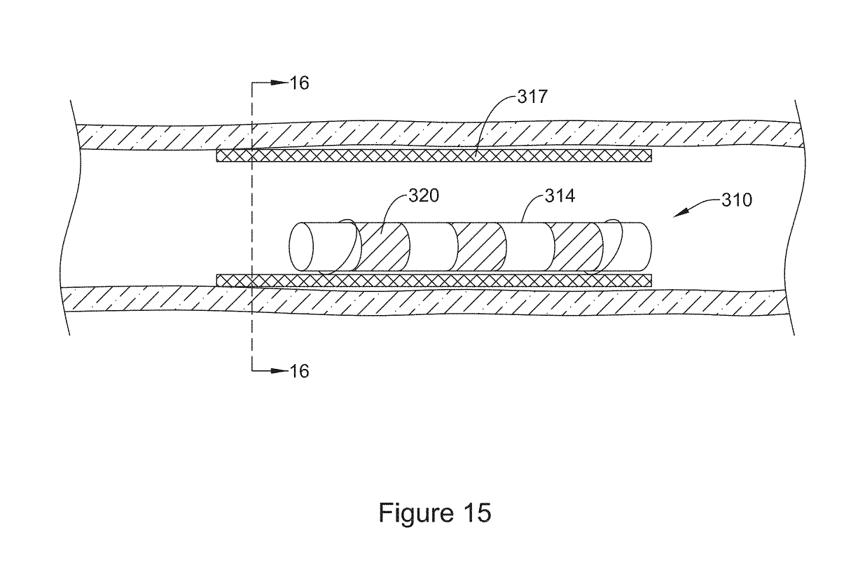

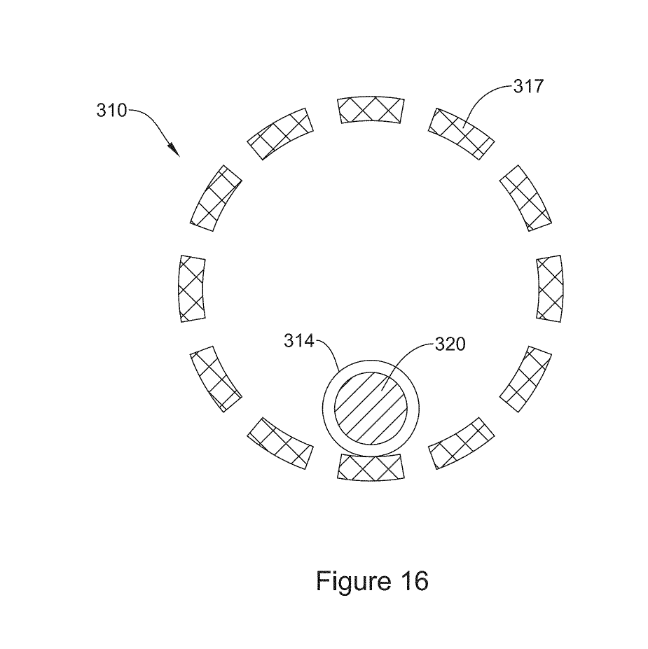

FIGS. 14 and 15 illustrate an example radioactive medical device 310 designed to shift along the longitudinal axis of medical device 310. As shown by the arrows in FIG. 14, attachment arms 316 may be designed such that they can flex, bend, rotate, deflect, etc. in a longitudinal direction, a radial direction, or both a longitudinal and radial direction. For example, FIG. 15 illustrates that in some examples frame member 314 may shift in a longitudinal direction such that frame 314 is positioned along anchoring member 317. FIG. 16 is a cross-section along line 16-16 of FIG. 15 illustrating the position of frame 314 (including radioactive elements 320) shifted such that frame 314 is positioned along anchoring member 317.

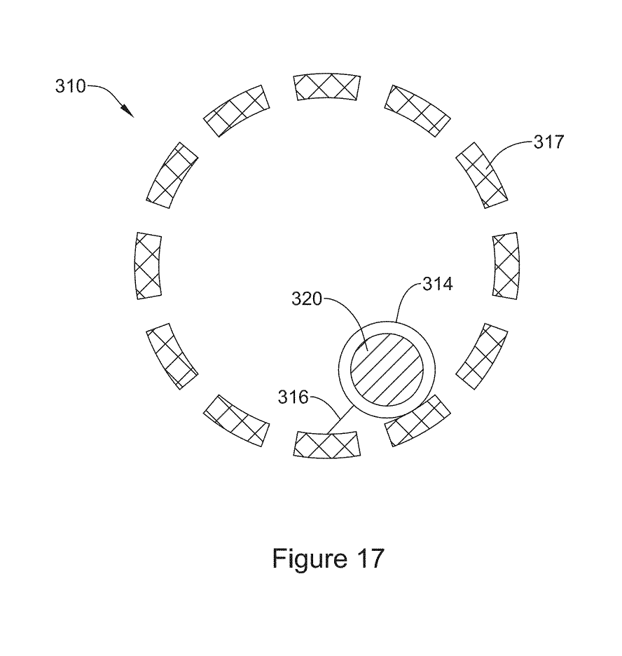

Additionally, while FIGS. 14-16 depict frame 314 (including radioactive elements 320) shifting along the longitudinal axis of medical device 310, some other examples contemplate frame 314 (including radioactive elements 320) shifting along the radial axis of radioactive medical device 310. FIG. 17 depicts frame member 314 shifting along the radial axis such that frame 314 (including radioactive elements 320) are positioned along anchoring member 317.

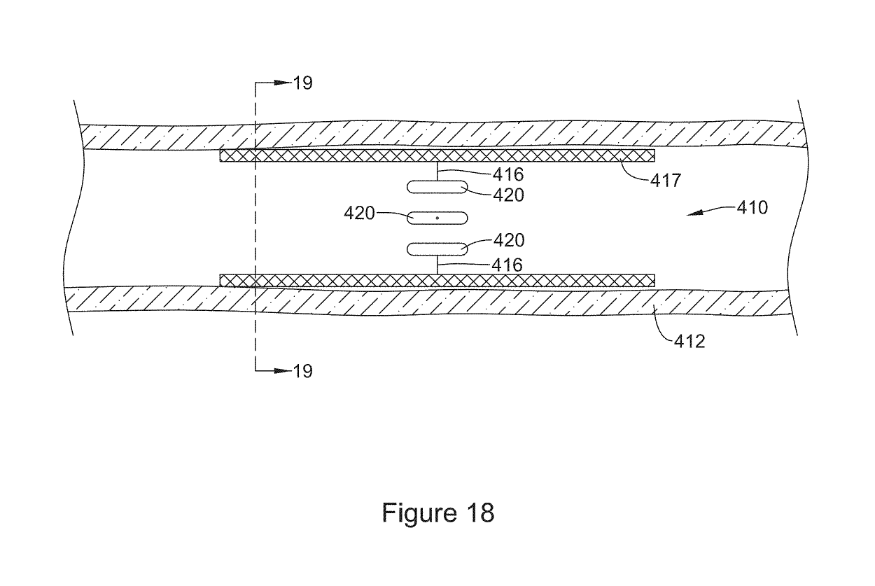

FIG. 18 illustrates another example radioactive medical device 410. As shown in FIG. 18, radioactive medical device 410 may include radioactive elements 420 and an anchoring member 417, which may be similar in structure to the radioactive elements and the anchoring members described in the examples above. Additionally, FIG. 18 further shows attachment arms 416 extending radially inward of the anchoring member 417.

Attachment arms 416 may be similar in structure to the attachment arms described in the examples above. The attachment arms 416 may be designed such that they position, suspend, locate, etc. the radioactive elements 420 in a central region of anchoring member 417 and example body lumen 412, such as around the central longitudinal axis of body lumen ix) 412. For example, attachment arms 416 may extend radially outward from the radioactive elements 420 to anchoring member 417 such that the radioactive elements 420 are centered around the central longitudinal axis of anchoring member 417, thereby positioning radioactive elements 420 in the central region of anchoring member 417 and body lumen 412, such as centrally positioning the radioactive elements 420 equidistant to the perimeter of the inner surface of the body lumen 412 (e.g., centering the radioactive elements 420 around the central longitudinal axis of anchoring member 417 and the body lumen 412).

It can be appreciated that positioning the radioactive elements 420 around the central axis of the body lumen 412 may permit fluids and other material to more easily flow through the medical device 410, while still permitting the radioactive elements to maintain a position within body lumen 412 to effectively treat the target tissue.

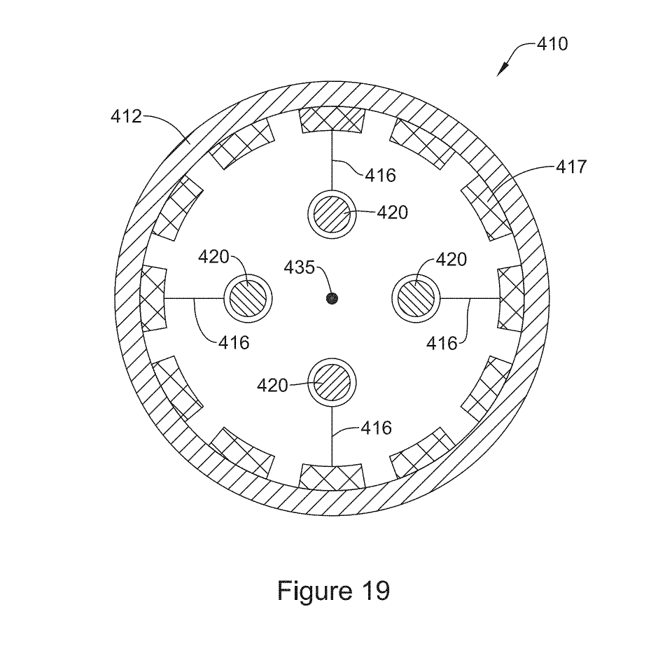

FIG. 19 is a cross-sectional view taken along line 19-19 of FIG. 18. FIG. 19 shows attachment arms 416 extending radially inward of the anchoring member 417. FIG. 19 further illustrates attachment arms 416 positioning the radioactive elements 420 in a central region of anchoring member 417 and example body lumen 412, such as around the central longitudinal axis 435 of body lumen 412.

It can be appreciated that FIG. 19 illustrates four support arms 416 spaced substantially equidistant around the central longitudinal axis 435 of body lumen 412. This configuration may position the four radioactive elements 420 substantially in a central region of the body lumen 412. However, it may be appreciated that radioactive device 410 may include more or less than four support members 416 and four radioactive elements 420. For example, radioactive device 410 may include 1, 2, 3, 4, 5, 6, 7, 8, 9, 10 or more support members 416 and 1, 2, 3, 4, 5, 6, 7, 8, 9, 10 or more radioactive elements 420. Furthermore, it can be appreciated that support members 416 may be spaced around example support member 417 in a variety of configurations and/or arrangements. Additionally, in other examples it is contemplated that there may be one or more rows of radioactive elements 420 spaced around support member 417 in a variety of configurations and/or arrangements.

In some instances, it may be desirable for a radioactive medical device to shift from a position in a central region of body lumen to a position in which the radioactive elements are closer to the inner surface of the an anchoring member (e.g., expandable stent member) in which it is positioned. For example, in some instances it may be desirable for medical device 410 (shown in FIG. 18 and FIG. 19) to be able to shift in response to being loaded into a medical device delivery system.

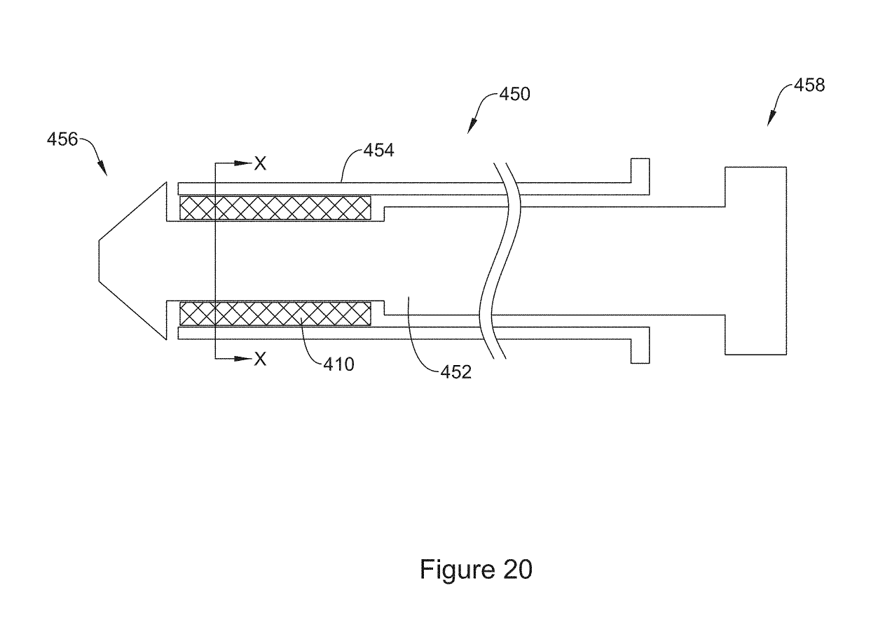

FIG. 20 illustrates an example medical device delivery system 450. Medical device delivery system 450 may include a distal end 456 and a proximal end 458. Medical device delivery system 450 may include an inner tubular member 452 and an outer tubular member 454. The inner tubular member 452 may extend through a lumen of the outer tubular member 454. In other words, the outer tubular member 454 may be disposed over the inner tubular member 452. Additionally, it can be appreciated that the outer tubular member 454 may be able to translate with respect to the inner tubular member 452. For example, the outer tubular member 454 may be able to translate (e.g., shift, slide, etc.) in a distal-to-proximal direction with respect to the inner tubular member 452.

FIG. 20 further illustrates the medical device 410 shown in FIG. 18 and FIG. 19 positioned between the outer tubular member 454 and the inner tubular member 452. It can be appreciated that the medical device 410 may be in a “pre-deployed” (e.g., “loaded”) configuration when positioned between the outer tubular member 454 and the inner tubular member 452. It can further be appreciated that translating the outer tubular member 454 in a distal-to-proximal direction may “release” (e.g., “deploy”) the medical device 410.

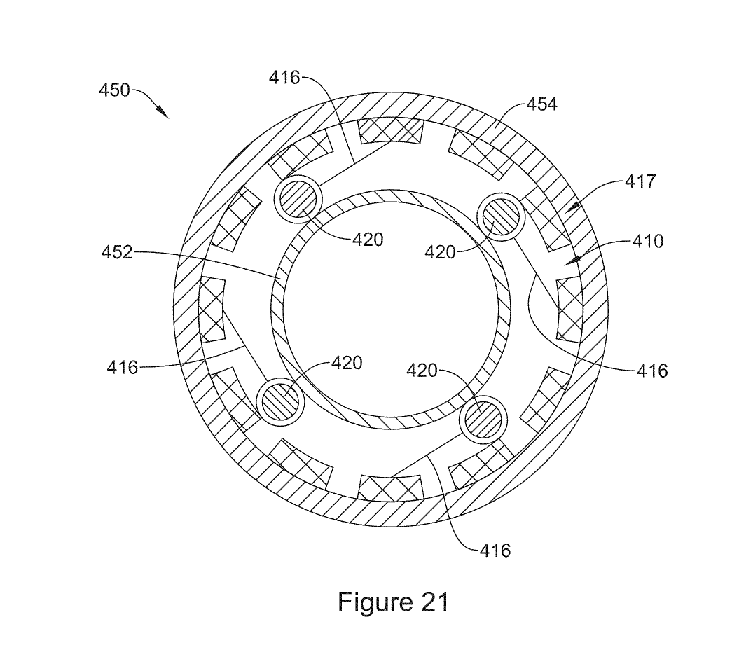

FIG. 21 is a cross-sectional view along line X-X of FIG. 20. FIG. 20 shows medical device 410 positioned between the outer tubular member 454 and the inner tubular member 452 in a pre-deployed (e.g., loaded) configuration. FIG. 21 further illustrates that attachment arms 416 may be designed such that they can flex, bend, rotate, deflect, etc. such that they move radially outward and closer to the inner surface of the anchoring member 417. For example, FIG. 21 illustrates that attachment arms 416 may shift such that the radioactive elements 420 may be adjacent to both the inner surface of the anchoring member 417 and the outer surface of the inner tubular member 452 of the medical device delivery system 450. It can further be appreciated that the medical device 410 (including the attachment arms 416 and the radioactive elements 420) may return to the configuration shown in FIG. 19 after being released (e.g., deployed) from the medical device delivery system 450.

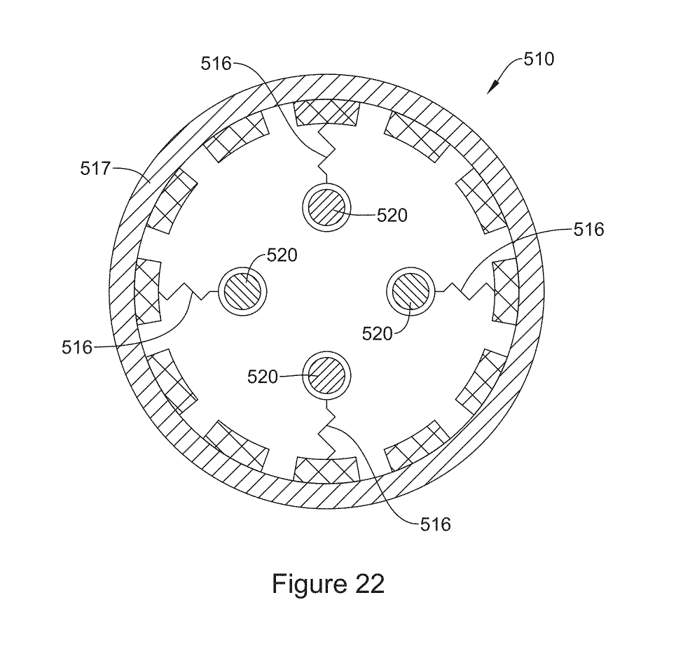

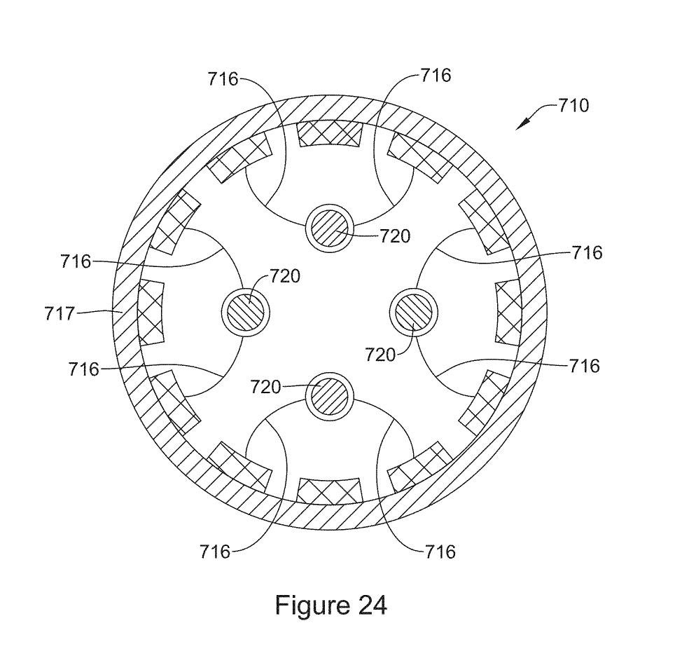

FIG. 22 illustrates another example radioactive medical device 510. As shown in FIG. 22, radioactive medical device 510 may include radioactive elements 520 and an anchoring member 517, which may be similar in structure to the radioactive elements and the anchoring members described in the examples above. However, FIG. 22 further shows attachment arms 516 extending radially inward of the anchoring member 517. Additionally, the attachment arms 516 shown in FIG. 22 may include a “zig-zag” configuration. As illustrated the, attachment arms 516 may include one or more bends and/or angles that may allow the attachment arms 516 to flex similar to a spring. While not shown in FIG. 22, it is contemplated that more than one attachment arm 516 may extend from the anchoring member to the radioactive element. An example of this configuration is shown in FIG. 24, however, the attachment arms shown in FIG. 24 are curved (versus the zig-zag configuration shown in FIG. 22).

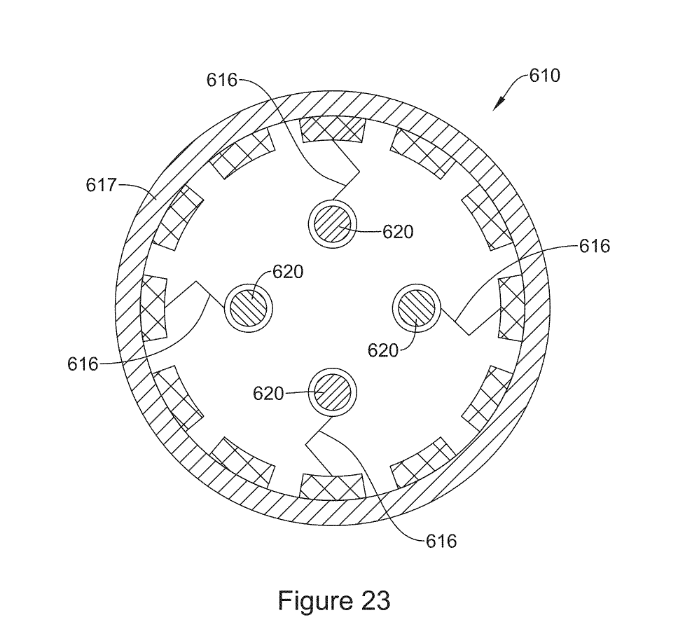

FIG. 23 illustrates another example radioactive medical device 610. As shown in FIG. 23, radioactive medical device 610 may include radioactive elements 620 and an anchoring member 617, which may be similar in structure to the radioactive elements and the anchoring members described in the examples above. However, FIG. 23 further shows attachment arms 616 extending radially inward of the anchoring member 617. Additionally, the attachment arms 616 shown in FIG. 23 may include a single “bent-arm” configuration. As illustrated the, attachment arms 616 may include a bend or angle that may allow the attachment arms 616 to flex similar to a spring. While not shown in FIG. 23, it is contemplated that more than one attachment arm 616 may extend from the anchoring member to the radioactive element. An example of this configuration is shown in FIG. 24, however, the attachment arms shown in FIG. 24 are curved (versus the bent-arm configuration shown in FIG. 23).

FIG. 24 illustrates another example radioactive medical device 710. As shown in FIG. 24, radioactive medical device 710 may include radioactive elements 720 and an anchoring member 717, which may be similar in structure to the radioactive elements and the anchoring members described in the examples above. However, FIG. 24 further shows attachment arms 716 extending radially inward of the anchoring member 717. Additionally, the attachment arms 716 shown in FIG. 24 may include one or more a curved arms attached to the radioactive elements 720. As illustrated the, attachment arms 720 may include a curved shape which may allow the attachment arms 716 to flex radially inward/outward.

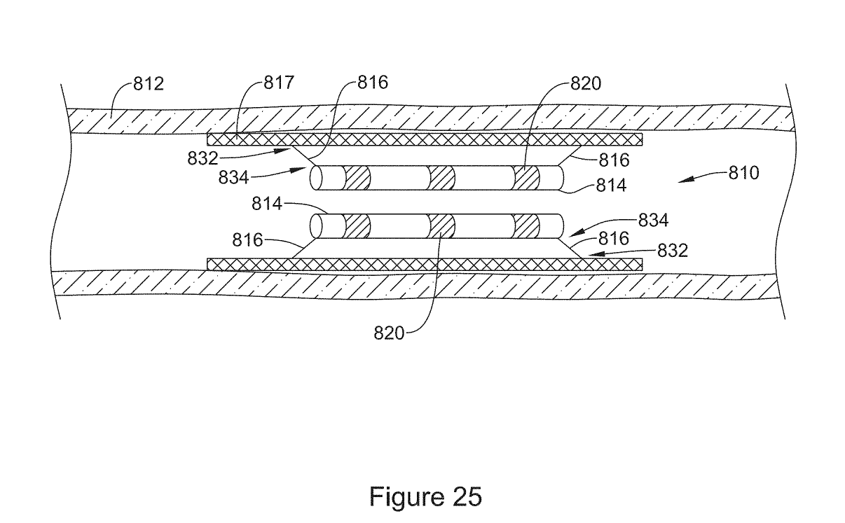

FIG. 25 illustrates another example radioactive medical device 810. Radioactive medical device 810 may include frame members 814, support members 816 and radioactive elements 820. Additionally, FIG. 25 illustrates an anchoring member 817 that substantially surrounds at least a portion of medical device 810. In some instances, anchoring member 817 may surround all of medical device 810. While FIG. 25 shows two frame members 814, it is contemplated that medical device 810 may include more or less than two frame members 814. For example, medical device 810 may include 1, 2, 3, 4, 5, 6 or more frame members 814 (including radioactive elements 820).

Anchoring member 817 may define a variety of designs and or structures. For example, anchoring member 817, such as a framework or scaffold, which may be an expandable framework or expandable scaffold in some instances, may include a stent, such as an expandable stent, a self-expanding stent, or another endoprosthesis or tubular member, for example. In some instances, anchoring member 817 may be manufactured from a single, cylindrical tubular member. For example, in some instances, a cylindrical tubular member may be laser cut to form an expandable stent. Anchoring member 817 may include one or more struts arranged in various designs and/or patterns. For example, anchoring member 817 may be a laser cut stent formed from a unitary tubular member. Therefore, numerous designs, patterns and/or configurations for the stent cell openings, strut thicknesses, strut designs, stent cell shapes are contemplated and may be utilized with embodiments disclosed herein. In other instances, anchoring member 817 may be a woven, braided or knitted tubular member, such as an expandable stent (e.g., self-expanding stent) formed from one or more, or a plurality of interwoven wire filaments.

As shown in FIG. 25, attachment arms 816 may extend away from the frame members 814 (including radioactive elements 820) and attach to anchoring member 817. For example, each of attachment arms 816 may include a first end 834 secured to frame member 814 and/or radioactive elements 820 and a second end 832 attached to anchoring member 817.

It can be appreciated that in some examples that medical device 810 may be configured to position the frame members 814 and radioactive elements 820 in a central region of body lumen 812, such as near the central longitudinal axis of body lumen 812. For instance, each of the support arms 816 may extend an equidistance between frame members ix) 814 and the anchoring member 817 such that frame members 814 (and radioactive elements 820) are centered along the central longitudinal axis of anchoring member 817, thereby positioning radioactive elements 820 in the central region of anchoring member 817 and body lumen 812, such as centrally positioning the radioactive elements 820 equidistant to the perimeter of the inner surface of the body lumen 812 (e.g., centering the radioactive elements 820 along the central longitudinal axis of anchoring member 817 and the body lumen 812).

It can be appreciated that positioning the frame members 814 and the radioactive members 820 around the central axis of the body lumen 812 may permit fluids and other material to more easily flow through the medical device 810, while still permitting the radioactive elements 820 to maintain a position within body lumen 812 to effectively treat the target tissue.

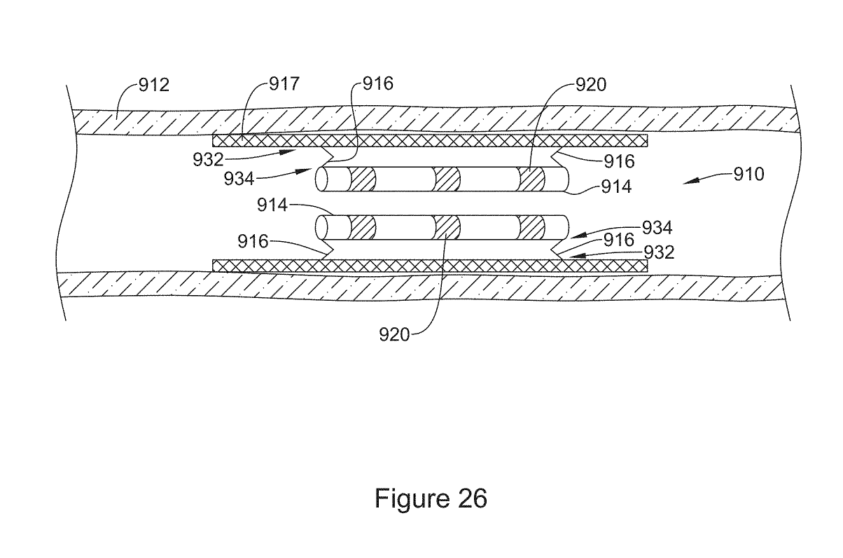

FIG. 26 illustrates another example radioactive medical device 910. As shown in FIG. 26, radioactive medical device 910 may include frame members 914 and radioactive elements 920 and an anchoring member 917, which may be similar in structure to the radioactive elements and the anchoring members described in the examples above. Additionally, the attachment arms 916 shown in FIG. 26 may include a single “bent-arm” configuration.

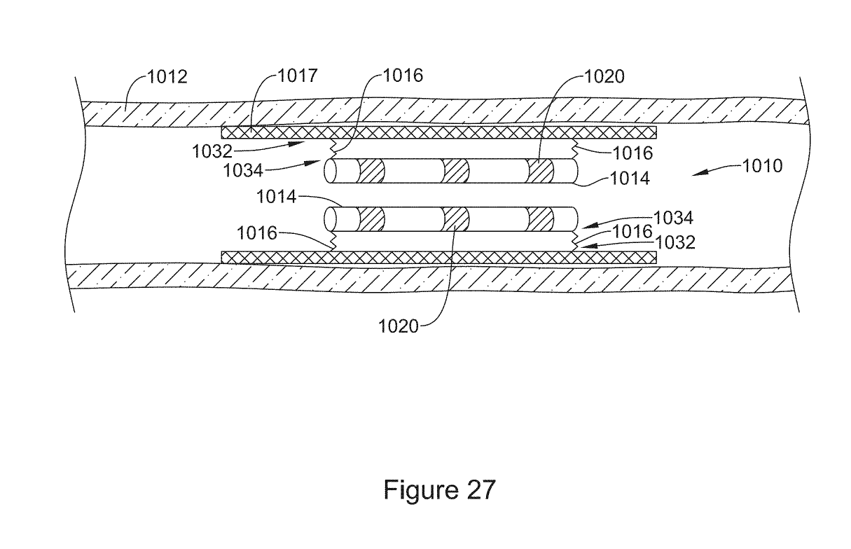

FIG. 27 illustrates another example radioactive medical device 1010. As shown in FIG. 27, radioactive medical device 1010 may include frame members 1014 and radioactive elements 1020 and an anchoring member 1017, which may be similar in structure to the radioactive elements and the anchoring members described in the examples above. Additionally, the attachment arms 1016 shown in FIG. 26 may include a “zig-zag” configuration including multiple bends.

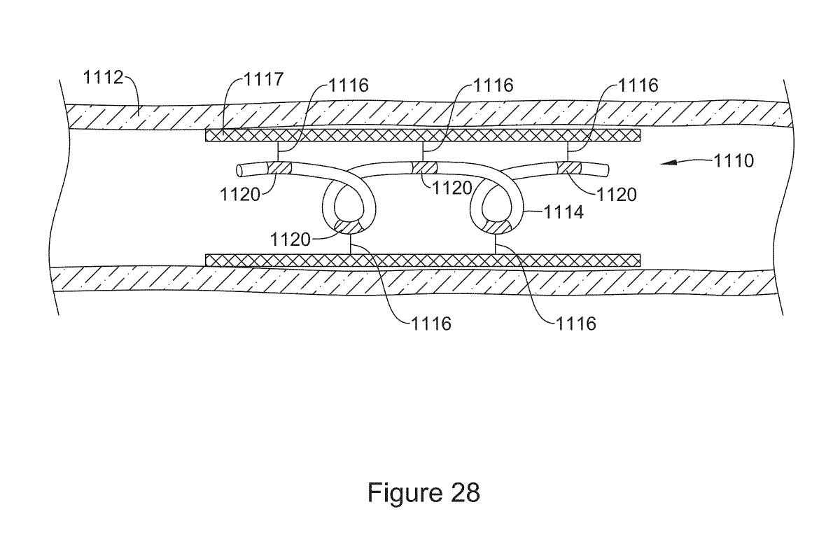

FIG. 28 illustrates another example radioactive medical device 1110. As shown in FIG. 28, radioactive medical device 1110 may include a spiral-shaped frame member 1114 and radioactive elements 1120 and an anchoring member 1117, which may be similar in structure to the radioactive elements and the anchoring members described in the examples above. Further, FIG. 28 shows attachment arms 1116 extending radially inward of the anchoring member 1117.

As discussed above, the frame member 1114 shown in FIG. 28 is substantially spiral shaped. For example, FIG. 28 illustrates the frame member 1114 including one or more curved portions extending along frame member 1114. It can be appreciated that a spiral-shaped frame member 1114 that is positioned around the central longitudinal axis of the body lumen 1112 may permit fluids and other material to more easily flow through the medical device 1110, while still permitting the radioactive elements 1120 to maintain a position within body lumen 1112 to effectively treat the target tissue. For example, FIG. 28 illustrates that the radioactive elements 1120 are spaced both around the central longitudinal axis of the body lumen 1112 and also spaced longitudinally along the body lumen 1112. This configuration may provide substantially evenly-spaced radioactive treatment to a length of the body lumen, while also permitting bodily fluids and material to pass through the medical device 1110, as discussed above.

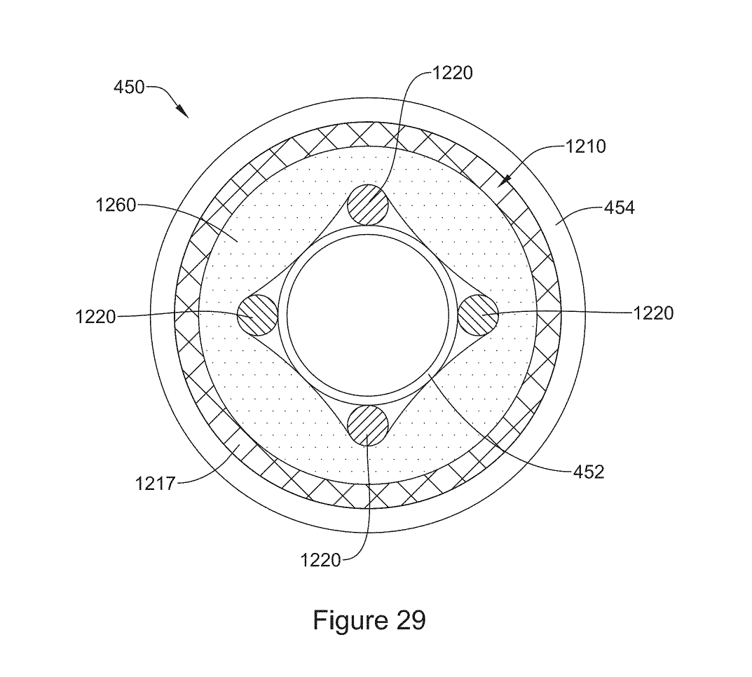

FIG. 29 illustrates another example radioactive medical device 1210. Similar to that described above, FIG. 29 shows a cross-sectional view of radioactive medical device 1210 positioned within the example medical device delivery system 450 (described above with respect to FIG. 20). For example, FIG. 29 shows medical device 1210 positioned between inner tubular member 452 and outer tubular member 454 in a pre-deployed (e.g., loaded) configuration.

As shown in FIG. 29, radioactive medical device 1210 may include radioactive elements 1220 and an anchoring member 1217, which may be similar in structure to the radioactive elements and the anchoring members described in the examples above. However, FIG. 29 further shows that medical device 1210 may include a foam spacer 1260 disposed between the inner tubular member 452 and the outer tubular member 454.

In at least some examples, the radioactive elements 1220 may be coupled (e.g., attached, etc.) to the foam spacer 1260. Further, while FIG. 29 shows the radioactive elements spaced substantially evenly around the foam spacer 1260, it is contemplated that the radioactive elements may be unevenly spaced around the foam spacer 1260. It is further contemplated that one or more of the radioactive elements 1220 may be at least partially positioned within the foam spacer 1260. For example, it is contemplated that one or more of the radioactive elements 1220 may be embedded within the foam spacer 1260. While FIG. 29 shows four radioactive elements 1220 positioned adjacent foam spacer 1260, it is contemplated that medical device 1210 may include more or less than four radioactive elements 1260. For example, medical device 1210 may include 1, 2, 3, 4, 5, 6, 7, 8, 9, 10 or more radioactive elements 1220.

As can be appreciated from FIG. 29, the foam spacer 1260 may be in a compressed configuration when positioned within the medical delivery device 450. In other words, in some examples the medical device 1210 (including the anchoring member 1217, foam spacer 1260 and radioactive elements 1220) may be compressed radially inward to permit loading of the medical device 1210 into the stent delivery device 450.

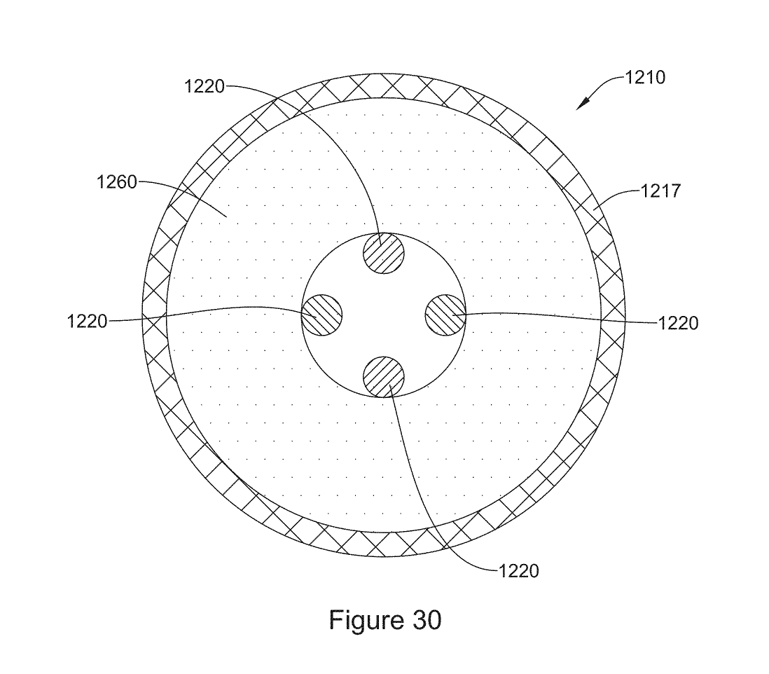

FIG. 30 illustrates the medical device 1210 in an expanded configuration. In other words, FIG. 30 illustrates medical device 1210 after having been deployed (e.g., released) from the medical device delivery system 450. As can be appreciated from FIG. 30, the anchoring member 1217 may expand radially outward after being released from the medical device delivery system 450. Additionally, in some examples, the foam spacer may expand radially outward, radially inward or both radially inward and radially outward.

It can be appreciated that in some examples that medical device 1210 may be configured to position the radioactive elements 1220 in a central region of a body lumen (not shown in FIG. 29 or FIG. 30), such as near the central longitudinal axis of a body lumen. For instance, the foam spacer 1260 may be centered around the central longitudinal axis of anchoring member 1217, thereby positioning radioactive elements 1220 in the central region of anchoring member 1217, such as centrally positioning the radioactive elements 1220 equidistant to the perimeter of the inner surface of a body lumen (e.g., centering the radioactive elements 1220 around the central longitudinal axis of anchoring member 1217 and the body lumen).

It can be appreciated that positioning the foam spacer 1260 and the radioactive members 1220 around the central axis of the body lumen may permit bodily fluids and other material to more easily flow through the medical device 1210, while still permitting the radioactive elements 1220 to maintain a position within body lumen 1212 to effectively treat the target tissue.

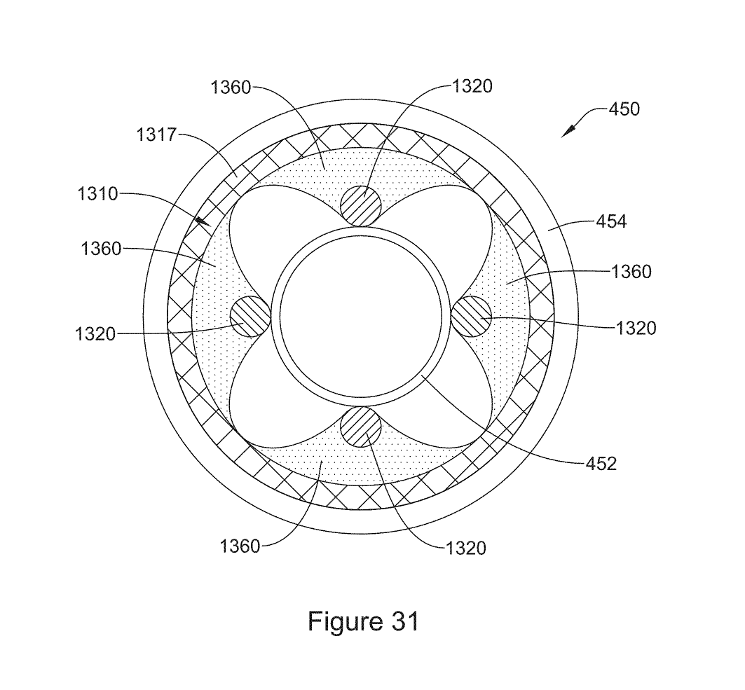

FIG. 31 illustrates another example radioactive medical device 1310. Similar to that described above with respect to FIG. 21, FIG. 31 shows a cross-sectional view of radioactive medical device 1310 positioned within the example medical device delivery system 450 (described above with respect to FIG. 20). For example, FIG. 31 shows medical device 1310 positioned between inner tubular member 452 and outer tubular member 454 in a pre-deployed configuration.

As shown in FIG. 31, radioactive medical device 1310 may include radioactive elements 1320 and an anchoring member 1317, which may be similar in structure to the radioactive elements and the anchoring members described in the examples above. However, FIG. 31 further shows that medical device 1310 may include a plurality of foam spacers 1360 disposed between the inner tubular member 452 and the outer tubular member 454.

In at least some examples, the radioactive elements 1320 may be coupled (e.g., attached, etc.) to the foam spacers 1360. Further, while FIG. 31 shows the radioactive elements 1320 spaced substantially evenly around the central longitudinal axis of the medical device 1310, it is contemplated that the radioactive elements 1320 may be unevenly spaced around the central longitudinal axis of the medical device 1310. It is further contemplated that one or more of the radioactive elements 1320 may be at least partially positioned within the foam spacers 1360. For example, it is contemplated that one or more of the radioactive elements 1320 may be embedded within the foam spacers 1360. While FIG. 31 shows four radioactive elements 1320 positioned adjacent four foam spacers 1360, it is contemplated that medical device 1310 may include more or less than four radioactive elements 1360 and four foam spacers 1360. For example, medical device 1310 may include 1, 2, 3, 4, 5, 6, 7, 8, 9, 10 or more radioactive elements 1320 and foam spacers 1360.

As can be appreciated from FIG. 31, the foam spacers 1360 may be in a compressed configuration when positioned within the medical delivery device 450. In other words, in some examples the medical device 1310 (including the anchoring member 1317, foam spacer 1360 and radioactive elements 1320) may be compressed radially inward to permit loading of the medical device 1310 into the stent delivery device 450.

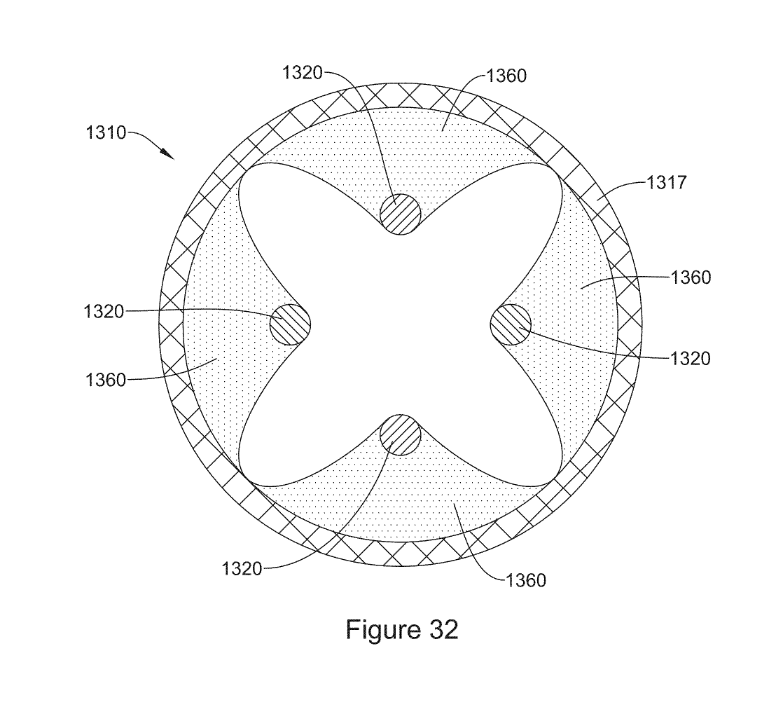

FIG. 32 illustrates the medical device 1310 in an expanded configuration. In other words, FIG. 32 illustrates medical device 1310 after having been deployed (e.g., released) from the medical device delivery system 450. As can be appreciated from FIG. 32, the anchoring member 1317 may expand radially outward after being released from the medical device delivery system 450. Additionally, in some examples, the foam spacers 1360 may expand radially outward, radially inward or both radially inward and radially outward.

It can be appreciated that in some examples that medical device 1310 may be configured to position the radioactive elements 1320 in a central region of a body lumen (not shown in FIG. 31 or FIG. 32), such as near the central longitudinal axis of a body lumen. For instance, the foam spacers 1360 may be centered around the central longitudinal axis of anchoring member 1317, thereby positioning radioactive elements 1320 in the central region of anchoring member 1317, such as centrally positioning the radioactive elements 1320 equidistant to the perimeter of the inner surface of a body lumen (e.g., centering the radioactive elements 1320 around the central longitudinal axis of anchoring member 1317 and the body lumen).

It can be appreciated that positioning the foam spacers 1360 and the radioactive members 1320 around the central axis of the body lumen may permit fluids (e.g., food or liquid) and other material to more easily flow through the medical device 1310, while still permitting the radioactive elements 1320 to maintain a position within the body lumen to effectively treat the target tissue.

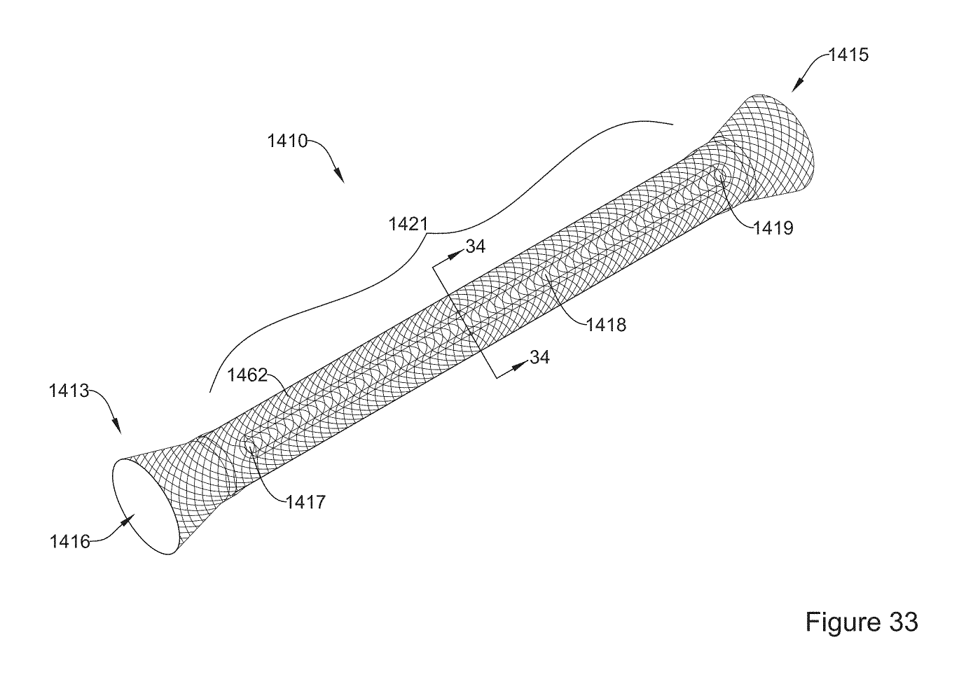

FIG. 33 shows another example anchoring device 1410. Medical device 1410 may include a stent member. Stent 1410 may include a plurality of filaments and/or strut members 1462 arranged in a variety of different designs and/or geometric patterns. For example, strut members 1462 may be a laser cut from a unitary tubular member. In other examples, filaments 1462 may be braided, woven, knitted or constructed using a combination of these (or similar) manufacturing techniques. Therefore, numerous designs, patterns and/or configurations for the stent cell openings, strut thicknesses, strut designs, stent cell shapes are contemplated and may be utilized with embodiments disclosed herein.

In some instances stent 1410 may be a self-expanding stent. A self-expanding stent may be delivered to a treatment area via a self-expanding stent delivery system. It is contemplated that the examples disclosed herein may be utilized with any one of various stent configurations, including, balloon expandable stents, such as a laser cut stent and/or a braided stent, a self-expanding stent, non-expandable stents, or other stents.

Stent filaments 1462 disclosed herein may be constructed from a variety of materials. For example, filaments 1462 may be constructed from a metal (e.g., Nitinol). In other instances, filaments 1462 may be constructed from a polymeric material (e.g., PET). In yet other instances, filaments 1462 may be constructed from a combination of metallic and polymeric materials. Additionally, filaments 1462 may include a bioabsorbable and/or biodegradable material.

Stent 1410 may include a first end region 1413, a second end region 1415 and a body portion 1421. Body portion 1421 may extend between the first end region 1413 and the second end region 1415. Further, stent 1421 may include a lumen 1416 extending within at least a portion of the stent 1410. Additionally, FIG. 33 illustrates that the first end region 1413, the second end region 1415 or both the first end region 1413 and the second end region 1415 may include a flared portion.

Further, FIG. 33 illustrates that the stent member 1410 may include one or more channels 1418 extending from the first end region 1413 to the second end region 1415. Each of the one or more channels 1418 may include a first end 1417 and a second end 1419. While FIG. 33 illustrates channels 1418 extending longitudinally along body portion 1421, this is not intended to be limiting. Rather, it is contemplated that channels 1418 may be longitudinal, helical, circumferential or any other of a variety of configurations along the stent member 1410.

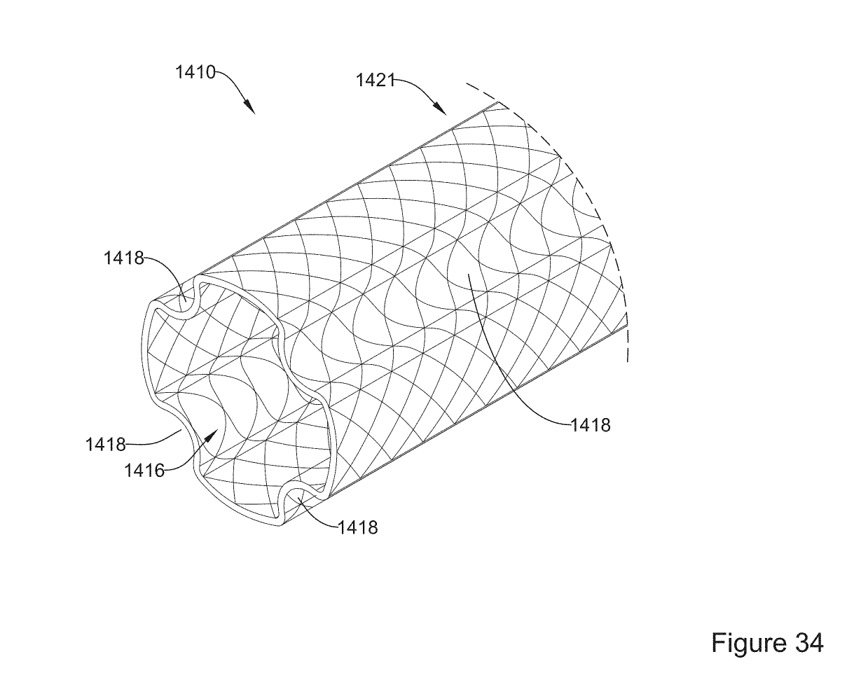

FIG. 34 is a cross-section along line 34-34 of FIG. 33. FIG. 34 illustrates four channels 1418 extending within the body portion 1421 of the stent 1410. While FIG. 34 illustrates four channels 1418 spaced around the circumference of the stent 1410, it is contemplated that stent 1410 may include more or less than four channels 1418. For example, stent 1410 may include 1, 2, 3, 4, 5, 6, 7, 8 or more channels 1418. Additionally, FIG. 34 illustrates that each of the channels 1418 may extend radially inward from the outer surface of the stent 1410.

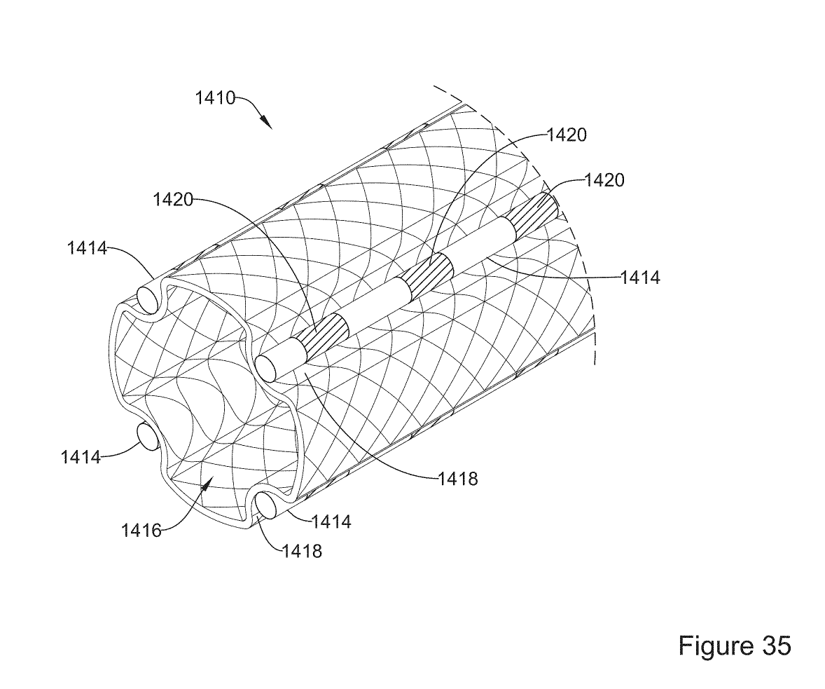

In some instances, it may be desirable to dispose a radioactive element along the stent member 1410. For example, FIG. 35 illustrates that stent member 1410 may include frame members 1414 and radioactive elements 1420 as described above. Further, FIG. 35 illustrates that the frame members 1414 may be disposed within the channels 1418 of the stent member 1410. Referring to FIG. 33, it is contemplated that the frame members 1414 (including the radioactive elements 1420) are positioned within the channels 1418 such that the ends of the frame members 1414 are adjacent the first end 1417 and the second end 1419 of each of the channels 1418. In other words, the first end 1417 and the second end 1419 may prevent the frame members 1414 from extending into the flared portions of the first end region 1413 and the second end region 1415 of the stent member 1410.

It can further be appreciated from FIG. 35 that the design of the stent member 1410 permits the frame members 1414 (including the radioactive elements 1420) to be held adjacent a target tissue site (via positioning within the channels 1418) while also permitting for body fluids and other material to flow through the lumen 1416 of the stent 1410.

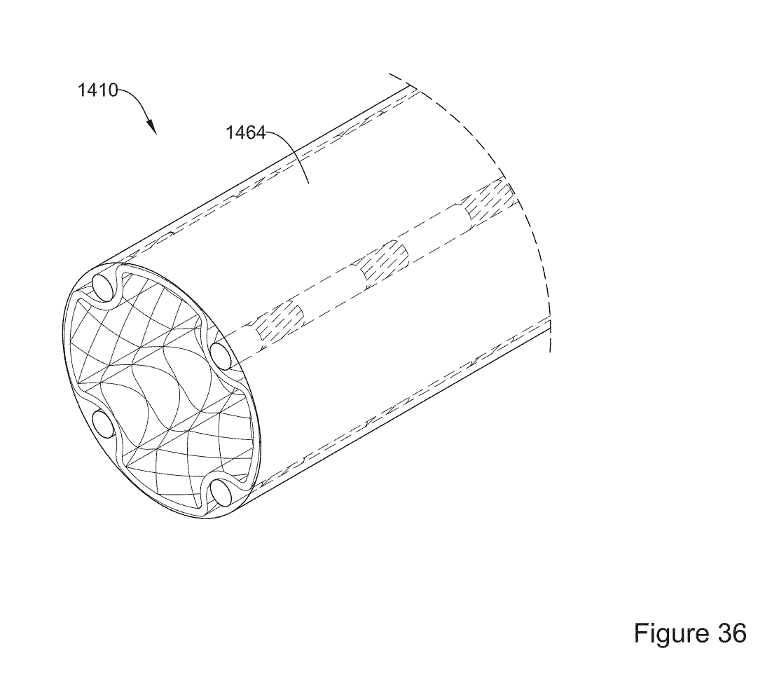

In some examples, stent 1410 may include a covering 1464. For example, FIG. 36 illustrates that stent 1410 may be partially or fully covered by an elastomeric or non-elastomeric material. Additionally, stent 1410 may be partially or fully covered by a polymeric material such as silicone or ePTFE. Further, the covering (e.g., polymer) 1464 may span the spaces (e.g., openings, cells) created by the geometric arrangements of filaments 1462.

It is further contemplated that in any of the examples disclosed herein, one or more structures of radioactive medical device 10 (or other examples thereof) may be designed to shield radioactive energy being emitted from radioactive elements 20 (or other examples thereof), thereby modulating the radiation delivered by radioactive elements. For example, frame 14 (or other examples thereof) and/or anchoring member 217 (or other examples thereof), may be designed such that they shield (e.g., modulate) the radioactive energy being emitted from radioactive elements 20 (or other examples thereof). For example, anchoring member 217 may include a stent strut (or stent strut pattern) that is designed to align with radioactive elements disclosed herein (for example, when radioactive elements 320 are shifted to be positioned along anchoring member 317) such that the stent struts themselves modulate the amount of radioactive energy being received by the target tissue.

Additionally, it is contemplated that in any of the example disclosed herein, all or a portion of frame 14 (or other examples thereof) and/or anchoring member 217 (or other examples thereof) may include a covering which incorporates a radiation shield, and thereby modulates the amount of radiation delivered by radiation elements positioned adjacent thereto.

Materials that may be used for the various components of the radioactive medical device 10 and the various examples disclosed herein may include those commonly associated with medical devices. For simplicity purposes, the following discussion makes reference to radioactive medical device 10. However, this is not intended to limit the devices and methods described herein, as the discussion may be applied to other similar systems and/or components of stent systems or devices disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device, comprising:
a radioactive element positionable within a body lumen, the body lumen having an inner surface; and
a frame attached to the radioactive element;
wherein the frame is configured to position the radioactive element radially inward and away from the inner surface of the body lumen.

2. The medical device of claim 1, wherein the frame is configured to position the radioactive element in a central region of the body lumen.

3. The medical device of claim 1, further comprising a plurality of radioactive elements, wherein the plurality of radioactive elements are attached together to form an elongated radioactive strand.

4. The medical device of claim 1, wherein the radioactive element is removably attached to the frame.

5. The medical device of claim 1, wherein the frame includes one or more support arms extending radially away from the radioactive element.

6. The medical device of claim 5, wherein each of the one or more support arms further comprises a spring member attached thereto.

7. The medical device of claim 5, wherein each of the one or more support arms are configured to be releasably engaged to the body lumen.

8. The medical device of claim 5, wherein the frame includes a retrieval member, and wherein pulling the retrieval member collapses each of the one or more support arms toward the radioactive element.

9. The medical device of claim 5, further comprising an anchoring member positioned around the radioactive element, and wherein each of the one or more support arms include a first end secured to the anchoring member.

10. The medical device of claim 9, the second ends of the one or more support arms are axially aligned.

11. The medical device of claim 10, wherein each of the one or more support arms are configured to permit the radioactive element to shift from a first position to a second position.

12. The medical device of claim 11, wherein shifting the support arms from a first position to a second position shifts the radioactive element in a radial direction, an axial direction, or both radial and axial directions.

13. The medical device of claim 5, further comprising a foam spacer secured between the radioactive element and the frame.

14. A medical device, comprising:
a support structure including a base member and one or more support members extending therefrom;
wherein the base member is configured to receive one or more radioactive elements;
wherein the base member is configured to position the one or more radioactive elements in a central region of a body lumen.

15. The medical device of claim 14, wherein the one or more radioactive elements are configured to be removed from the support structure.

16. The medical device of claim 14, wherein each of the one or more support members are configured to be releasably engaged to the body lumen.

17. The medical device of claim 14, further comprising an anchoring member positioned around the support structure, and wherein each of the one or more support members include a first end secured to the base member and a second end secured to the anchoring member.

18. The medical device of claim 14, wherein each of the one or more support members are configured to permit the base member to shift from a first position to a second position.

19. The medical device of claim 18, wherein shifting the one or more support members from a first position to a second position shifts the base member in both a radial direction, an axial direction, or both radial and axial directions.

20. A medical device, comprising:
a radioactive element; and
an expandable scaffold including a base member and one or more support members extending radially from the base member, wherein each of the one or more support members has a first end attached to the scaffold and a second end attached to the base member;
wherein the base member is configured to receive the radioactive element;
wherein the one or more support members is/are configured to suspend the base member in a central region of a body lumen.
